(19) United States
(12) Patent Application Publication
(10) Pub. No.: US 2017/0296450 A1
(43) Pub. Date: Oct. 19, 2017

(54) METHODS AND MATERIALS FOR DELIVERING AGENTS TO HAIR, SKIN, OR NAILS

(71) Applicant: SAFEWHITE, INC., Columbus, OH (US)

(72) Inventors: Richard Simon Brody, Columbus, OH (US); Scott Joseph Bridgeman, Delaware, OH (US); Uday B. Sandbhor, Hilliard, OH (US); Nicole Hoefer, Bexley, OH (US)

(73) Assignee: SAFEWHITE, INC., Columbus, OH (US)

(21) Appl. No.: 15/511,787

(22) PCT Filed: Sep. 17, 2015
(86) PCT No.: PCT/US15/50668

§ 371 Date: Mar. 16, 2017

Related U.S. Application Data
(60) Provisional application No. 62/051,723, filed on Sep. 17, 2014.

(51) Int. Cl. A61K 8/44; A61K 8/64; A61Q 3/02; A61Q 5/06; A61K 8/65; A61K 8/19; A61K 8/81; A61K 8/60; A61Q 19/04

(52) U.S. Cl. CPC A61K 8/44; A61K 8/602; A61K 8/64; A61Q 3/02; A61Q 19/04; A61K 8/65; A61K 8/19; A61K 8/817; A61K 8/8152; A61Q 5/065; A61K 2800/882; A61K 2800/884; A61K 2800/43

(57) ABSTRACT

This document provides methods and materials for delivering agents to hair, skin, and/or nails using one or more adhesive molecules alone or in combination with one or more interlocking metals, one or more dyes, and/or one or more polypeptides. For example, methods and materials for delivering interlocking metals, dyes, or polypeptides to hair, skin, or nails of a mammal (e.g., a human) are provided.

### BACKGROUND

### 1. Technical Field

This document relates to methods and materials for delivering agents to hair, skin, and/or nails using one or more adhesive molecules. This document also relates to altering the color, appearance, or stability of hair, skin, and/or nails. For example, this document relates to methods and materials for contacting hair, skin, and/or nails with one or more adhesive molecules (e.g., one or more mussel adhesive polypeptides) in combination with interlocking metals (e.g., copper), dyes (e.g., carmine), and/or polypeptides (e.g., keratin or albumin polypeptides).

### 2. Background Information

People color their hair, skin, and/or nails for a variety of reasons. The main reasons are cosmetic (e.g., to cover gray hair or to change one's hair, skin, or nails to a color regarded as more fashionable or desirable).

### SUMMARY

This document provides methods and materials for delivering agents to hair, skin, and/or nails using one or more adhesive molecules alone or in combination with one or more interlocking metals, one or more dyes, and/or one or more polypeptides. For example, the methods and materials described herein can be used to deliver interlocking metals, dyes, or polypeptides to the hair, skin, or nails of a mammal (e.g., a human). In some cases, delivery of an agent described herein can improve the appearance of the hair, skin, or nails, can alter the appearance of the hair, skin, or nails, or can strengthen the integrity of the hair, skin, or nails.

This document also provides methods and materials for altering the color of hair, skin, or nails of a mammal (e.g., a human). For example, this document provides methods and materials for contacting hair, skin, or nails with one or more adhesive molecules (e.g., one or more mussel adhesive polypeptides) in combination with one or more interlocking metals and/or one or more dyes to provide the hair, skin, or nails with an altered color appearance. As described herein, adhesive molecules can be applied to hair, skin, or nails in combination with interlocking metals, dyes, and/or polypeptides. The adhesive molecule can include a plurality of 3,4-dihydroxyphenyl-L-alanine (DOPA) residues and can have the ability to interact with or bind to hair, skin, and/or nails as well as the ability to interact with or bind to interlocking metals, dyes, and/or polypeptides. The methods and materials described herein can be used on dry hair, skin, and/or nails or on wet or moist hair, skin, and/or nails.

In general, one aspect of this document features a composition comprising, or consisting essentially of, an adhesive molecule comprising a plurality of 3,4-dihydroxyphenyl-L-alanine (DOPA) residues, and metal ions or a dye attached to the adhesive molecule via an interaction with one or more of the DOPA residues, wherein the adhesive molecule comprises the ability to interact with or bind to the hair, skin, or nails of a mammal. The adhesive molecule can be a mussel adhesive polypeptide. The adhesive molecule can be a polymer comprising a plurality of lysine residues and the plurality of DOPA residues. The adhesive molecule can be a polymer comprising a plurality of lysine residues, a plurality of glycine residues and the plurality of DOPA residues. The adhesive molecule can be a polymethacrylate polymer comprising the plurality of DOPA residues. The composition can comprise the metal ions. The metal ions can be copper ions, bismuth ions, chromium ions, iron ions, silver ions, aluminum ions, manganese ions, zinc ions, or combinations thereof The composition can comprise the dye. The dye can be carmine, henna, guanine, pyrophyllite, or mica. The composition can further comprise a polypeptide. The polypeptide can be a keratin polypeptide or fluorescence emitting polypeptide. The polypeptide can be conjugated to the adhesive molecule. The composition can be a shampoo, hair conditioner, gel, polish, or paste.

In another aspect, this document features a method for altering the appearance of hair, skin, or nails of a mammal. The method comprises, or consists essentially of, applying any composition of the preceding paragraph or any composition provided herein to hair, skin, or nails, wherein one or more of the DOPA resides interact with the hair, skin, or nails, and wherein the appearance of the hair, skin, or nails is altered. The composition can be applied to hair. The composition can be applied to skin. The composition can be applied to nails.

In another aspect, this document features a method for altering the appearance of hair, skin, or nails. The method comprises, or consists essentially of, (a) applying an adhesive molecule comprising a plurality of DOPA residues to hair, skin, or nails, wherein one or more of the DOPA resides interact with the hair, skin, or nails, and (b) applying metal ions or a dye to the hair, skin, or nails, wherein the metal ions or the dye interact with one or more of the DOPA resides of the adhesive molecule, wherein the appearance of the hair, skin, or nails is altered. The adhesive molecule and the metal ions or the dye can be applied sequentially. The adhesive molecule and the metal ions or the dye can be applied together. The adhesive molecule can be selected from the group consisting of a polymethacrylate polymer, the polymer comprising the plurality of DOPA residues; a mussel adhesive polypeptide; a polymer comprising a plurality of lysine residues and the plurality of DOPA residues; and a polymer comprising a plurality of lysine residues, a plurality of glycine residues, and the plurality of DOPA residues. The adhesive molecule can be a mussel adhesive polypeptide. The adhesive molecule can be a polymer comprising a plurality of lysine residues and the plurality of DOPA residues. The adhesive molecule can be a polymer comprising a plurality of lysine residues, a plurality of glycine residues and the plurality of DOPA residues. The adhesive molecule can be a polymethacrylate polymer comprising the plurality of DOPA residues. The method can comprise applying the metal ions to the hair, skin, or nails. The metal ions can be copper ions, bismuth ions, chromium ions, iron ions, silver ions, aluminum ions, manganese ions, zinc ions, or combinations thereof The method can comprise applying the dye to the hair, skin, or nails. The dye can be carmine, henna, guanine, pyrophyllite, or mica. The method can further comprise applying a polypeptide to the hair, skin, or nails. The polypeptide can be a keratin polypeptide or fluorescence emitting polypeptide. The polypeptide can be conjugated to the adhesive molecule.

In another aspect, this document features a delivery film comprising, or consisting essentially of, a lyophilized mixture of 3,4-dihydroxyphenyl-L-alanine (DOPA) and a polymer. The polymer can be PMA, PEMA, and PBMA. The delivery film can comprise an agent. The agent can be an interlocking metal, a dye, a polypeptide, a fluorescent molecule, an antibiotic, a therapeutic agent, a whitening particle, or a coloring particle.

In another aspect, this document features a method for delivering an agent to hair, skin, or nails of a mammal. The method comprises, or consists essentially of, (a) contacting a delivery film to the hair, skin, or nails, and (b) contacting the delivery film with the agent. The delivery film comprises, or consists essentially of, a lyophilized mixture of 3,4-dihydroxyphenyl-L-alanine (DOPA) and a polymer. The polymer can be PMA, PEMA, and PBMA. The agent can be an interlocking metal, a dye, a polypeptide, a fluorescent molecule, an antibiotic, a therapeutic agent, a whitening particle, or a coloring particle.

In another aspect, this document features a method for delivering an agent to hair, skin, or nails of a mammal, wherein the method comprises contacting a delivery film to the hair, skin, or nails, thereby delivering the agent to the hair, skin, or nails. The delivery film comprises, or consists essentially of, a lyophilized mixture of 3,4-dihydroxyphenyl-L-alanine (DOPA) and a polymer and further comprises an agent. The polymer can be PMA, PEMA, and PBMA. The agent can be an interlocking metal, a dye, a polypeptide, a fluorescent molecule, an antibiotic, a therapeutic agent, a whitening particle, or a coloring particle.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a listing of the nucleic acid sequence (SEQ ID NO:1) that encodes an exemplary mussel adhesive polypeptide (GenBank Accession No. AY521220.1; GI No. 41350294).

FIG. 2 is a listing of an amino acid sequence (SEQ ID NO:2) of an exemplary mussel adhesive polypeptide (GenBank Accession No. AAS00463; GI No. 41350295).

FIG. 3 is a listing of an amino acid sequence (SEQ ID NO:3) of an exemplary mussel adhesive polypeptide (GenBank Accession No. AAL35297.1; GI No. 17066511).

FIG. 4 is a listing of an amino acid sequence (SEQ ID NO:4) of an exemplary mussel adhesive polypeptide (GenBank Accession No. ABE01084.1; GI No. 90823165).

FIG. 5 is a listing of an amino acid sequence (SEQ ID NO:5) of an exemplary mussel adhesive polypeptide (GenBank Accession No. AAF89290.1; GI No. 9587380).

FIG. 6 is a listing of an amino acid sequence (SEQ ID NO:6) of an exemplary mussel adhesive polypeptide (GenBank Accession No. AAY29129.1; GI No. 63055693).

FIG. 7 is a listing of an amino acid sequence (SEQ ID NO:7) of an exemplary mussel adhesive polypeptide (GenBank Accession No. BAB16314.1; GI No. 10641127).

FIG. 8 is a listing of an amino acid sequence (SEQ ID NO:8) of an exemplary mussel adhesive polypeptide (GenBank Accession No. AAX23968.1; GI No. 60548042).

FIG. 9 is a listing of an amino acid sequence (SEQ ID NO:9) of an exemplary mussel adhesive polypeptide (GenBank Accession No. AAY29131.1; GI No. 63055728).

FIG. 10 is a listing of a nucleic acid sequence (SEQ ID NO:10) that encodes an exemplary BFP polypeptide (GenBank Accession No. U70497.1; GI No. 1619752).

FIG. 11 is a listing of an amino acid sequence (SEQ ID NO:11) of an exemplary BFP polypeptide (GenBank Accession No. AAB16959.1; GI No. 1619753).

FIG. 12 is a listing of an amino acid sequence (SEQ ID NO:12) of an exemplary BFP polypeptide.

### DETAILED DESCRIPTION

This document provides methods and materials for using an adhesive molecule comprising a plurality of DOPA residues to adhere another compound (e.g., an interlocking metal, a dye, a polypeptide, a fluorescent molecule, a polymer, an antibiotic, a therapeutic agent, a nucleic acid, a whitening particle, a coloring particle, a rejuvenating particle, a color-changing pigment, a composite pigment, a glow-in-the-dark coloring agent, a silica coated particle, a liquid crystal color, a tattoo pigment, a theoretical makeup, or a biological moiety) to hair, skin, and/or nails. In some cases, this document provides methods and materials for attaching agents to hair, skin, and/or nails for providing an altered appearance, for delivering an anti-bacterial agent, or for providing a therapeutic or aesthetic use. For example, this document provides methods and materials for contacting hair with an adhesive molecule and an interlocking metal or a dye to provide the hair with an altered color appearance.

As used herein a “DOPA residue” can include, for example, a small synthetic DOPA peptide and/or a catechol polymer that mimics DOPA/Lys (see, e.g, Ham et al. Angew. Chem. Int. Ed. 50:732-736 (2011)).

An adhesive molecule provided herein can include a plurality of DOPA residues and can have the ability to interact with or bind to hair, skin, and/or nails as well as the ability to interact with or bind to other compounds (e.g., an interlocking metal, a dye, a polypeptide, a fluorescent molecule, a polymer, an antibiotic, or a therapeutic agent, a nucleic acid, a whitening particle, a rejuvenating particle, or a biological moiety). Examples of adhesive molecules that can be used as described herein include, without limitation, mussel adhesive polypeptides (e.g., mussel foot proteins 1, 2, 3, 4, 5, 6, or combinations thereof). Mussel adhesive polypeptides can include one or more DOPA residues formed, for example, via enzymatic oxidation of tyrosine residues. For example, 1, 2, 3, 4, 5, 6, 7, 8, 9, 10, 15, 20, 25 percent or more of the total amino acids of a mussel adhesive polypeptide can be DOPA residues. The tyrosine residues of a recombinant polypeptide can be converted to DOPA residues using a tyrosinase (e.g., a mushroom tyrosinase). See, e.g., Choi et al., Microb. Cell. Fact., 11:139 (2012).

For example, an adhesive molecule can have at least 80%, 85%, 90%, 95%, 98%, or 99% sequence identity to any one of the amino acid sequences set forth in SEQ ID NOs:2-9 and 13-29. In some cases, an adhesive molecule can have the amino acid sequence set forth in SEQ ID NOs:2-9 and 13-29. The percent identity between a particular amino acid sequence and the amino acid sequence set forth in any one of

SEQ ID NOs:2-9 and 13-29 can be determined as follows. First, the amino acid sequences are aligned using the BLAST 2 Sequences (B12seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the B12seq program can be found in the readme file accompanying BLASTZ. B12seq performs a comparison between two amino acid sequences using the BLASTP algorithm. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences. Similar procedures can be followed for nucleic acid sequences except that blastn is used.

Once aligned, the number of matches is determined by counting the number of positions where an identical amino acid residue is presented in both sequences. The percent identity is determined by dividing the number of matches by the length of the amino acid sequence in any one of SEQ ID NOs:2-9 and 13-29, followed by multiplying the resulting value by 100.

It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

It will be appreciated that a number of nucleic acids can encode the amino acid sequences set forth in SEQ ID NOs:2-9 and 13-29. The degeneracy of the genetic code is well known to the art; i.e., for many amino acids, there is more than one nucleotide triplet that serves as the codon for the amino acid.

A mussel adhesive polypeptide that can be used as described herein can have an amino acid sequence that is naturally occurring in any type of mussel. For example, a mussel adhesive polypeptide that can be used as described herein can have an amino acid sequence that is naturally occurring in Mytilus edulis (common blue mussel), Mytilus byssus, Mytilus galloprovincialis, Mytilus californianus, Mytilus coruscus, Mytilus trossulusor, or Perna viridis (green mussel). For example, a mussel adhesive polypeptide that can be used as described herein includes, without limitation, mfp-5 from Mytilus galloprovincialis (GenBank Accession No. AAS00463), Mytilus edulis (GenBank Accession No. AAL35297.1), or Mytilus californianus (GenBank Accession No. ABE01084.1); mfp-3 from Mytilus edulis (GenBank Accession No. AAF89290.1, mfp-3 precursor variant 11), Mytilis californianus (GenBank Accession No. AAY29129.1) or Mytilus galloprovincialis (GenBank Accession No. BAB16314.1); or mfp-1 from Mytilus edulis (GenBank Accession No., AAX23968.1), Mytilis californianus (GenBank Accession No. AAY29131.1), or Mytilus galloprovincialis (UniProtKB/Swiss-Prot Q27409.1), or a fragment of any of the naturally-occurring mussel adhesive polypeptides.

In some cases, a mfp-1 mussel adhesive polypeptide can include one or more copies of a consensus sequence such as AKPSYPPTYK (SEQ ID NO:13) or PKISYPPTYK (SEQ ID NO:14). For example, a mussel adhesive polypeptide can include 5, 10, 15, 20, 25, 30, 35, 40, 45, 50, 55, 60, 65, 70, or 75 repeats of the consensus sequences set forth in SEQ ID NO:13 or SEQ ID NO:14. In some cases, the proline residues at position 6 and/or 7 are hydroxyproline residues. In some cases, the tyrosine residues at positions 5 and/or 9 are DOPA residues. In some cases, a mfp-2 mussel adhesive polypeptide can include one or more copies of a consensus sequence such as TDKAYKPNPCVVSKPCKNRGKCIWNGKAYRCKCAYGYGGRHC (SEQ ID NO:15). For example, a mussel adhesive polypeptide can include 2, 4, 5, 6, 7, 8, 9, 10, or 11 repeats of the consensus sequences set forth in SEQ ID NO:15. In some cases, the tyrosine residues at positions 5, 29, 35, and/or 37 of SEQ ID NO:15 are DOPA residues.

In some cases, a mfp-3 mussel adhesive polypeptide can include one or more copies of a consensus sequence such as ADYYGPNYGPPRRYGGGNYNRYNRYGRRYGGYKGWNNGWNRGRRGKYW (SEQ ID NO:16). In some cases, the tyrosine residues at positions 3, 4, 8, 14, 19, 22, 25, 29, 32, and/or 47 of SEQ ID NO:16 are DOPA residues.

In some cases, a mfp-4 mussel adhesive polypeptide can include one or more copies of a consensus sequence such as HVHTHRVLHK (SEQ ID NO:17) or DDHVNDIAQTA (SEQ ID NO:18). For example, a mussel adhesive polypeptide can include 5, 10, 12, 14, 16, 20, 25, 30, 32, 34, 35, or 36 repeats of the consensus sequences set forth in SEQ ID NO:17 or SEQ ID NO:18.

In some cases, a mfp-5 mussel adhesive polypeptide can include one or more copies of a consensus sequence such as SSEEYKGGYYPGNAYHYSGGSYHGSGYHGGYKGKYYGKAKKYYYKYKNSGKYKYLKKARKYHRKGYKYYGG SS (SEQ ID NO:19). In some cases, the tyrosine residues at positions 5, 9, 10, 15, 17, 22, 27, 31, 35, 36, 42, 43, 44, 46, 52, 54, 61, 66, 68, and/or 69 of SEQ ID NO:19 are DOPA residues.

In some cases, a mfp-6 mussel adhesive polypeptide can include one or more copies of a consensus sequence such as GGGNYRGYCSNKGCRSGYIFYDNRGFCKYGSSSYKYDCGNYACLPRNPYGRVKYYCTKKYSCPDDFYYYNNKGY YYYNDKDYGCFNCGSYNGCCLRSGY (SEQ ID NO:20). In some cases, the tyrosine residues at positions 5, 8, 18, 21, 29, 34, 36, 41, 49, 54, 55, 60, 67, 68, 69, 74, 75, 76, 77, 82, 90, and/or 99 of SEQ ID NO:20 are DOPA residues.

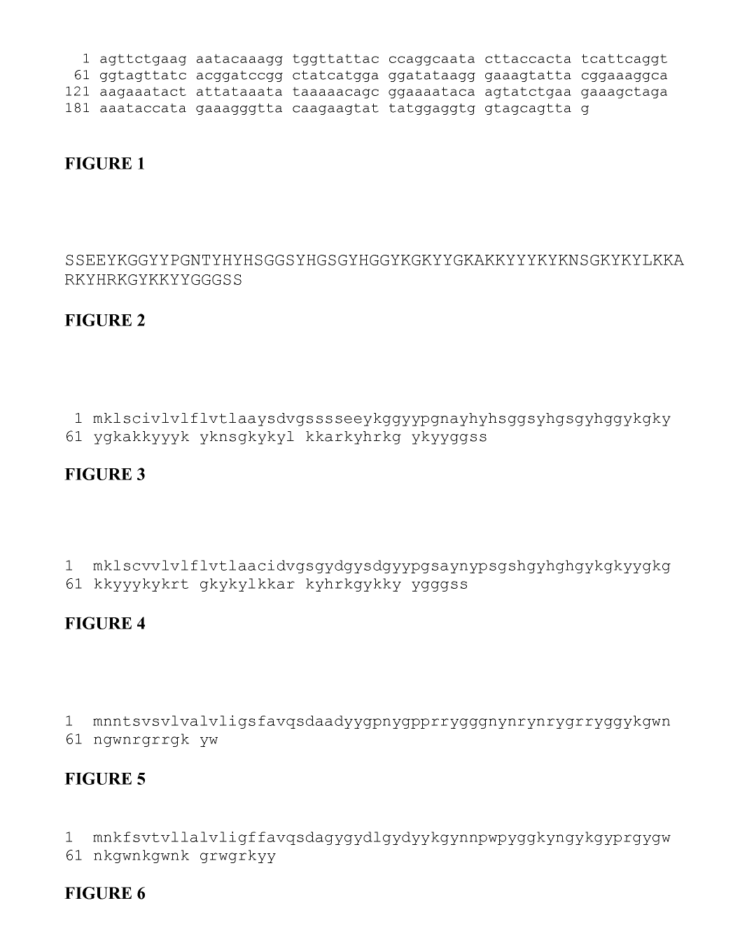

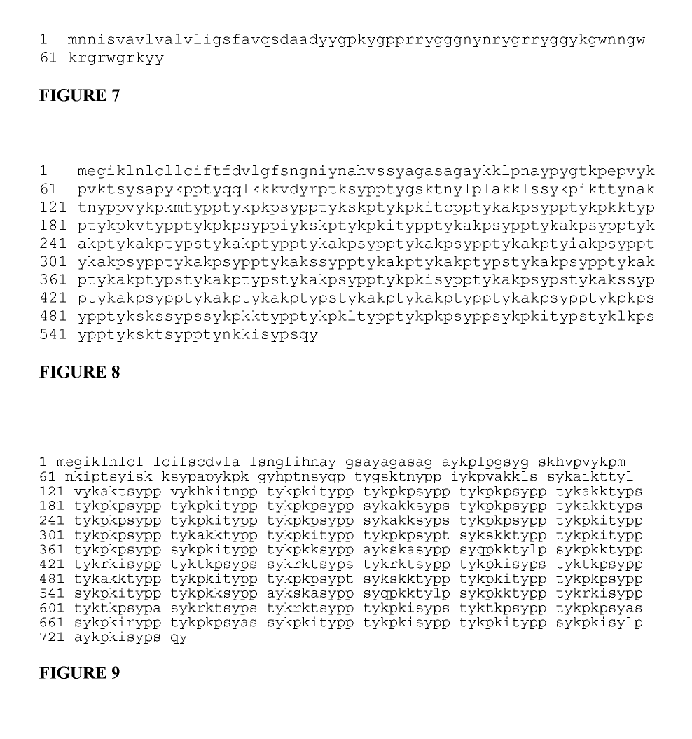

In some cases, a mussel adhesive polypeptide having the amino acid sequence set forth in any one of FIGS. 2-9 (SEQ ID NOs:2-9) or having an amino acid sequence encoded by the nucleotide sequence set forth in FIG. 1 can be used as described herein.

In some cases, a mussel adhesive polypeptide can be a portion of a full-length mussel adhesive polypeptide. For example, a mussel adhesive polypeptide can be used as described herein that includes six repeats of the decapeptide AKPSYPPTYK (SEQ ID NO:13). See, Kitamura et al., J. Polymer Science: Part A: Polymer Chemistry, 37:729-736 (1991).

In some cases, a mussel adhesive polypeptide that can be used as described herein can be a chimeric polypeptide that includes six decapeptide (AKPSYPPTYK; SEQ ID NO:13) repeats of MFP-1 at both the N- and C-termini of MFP-3 (e.g., SEQ

ID NO:8). See, Lim, et al., Biomaterials, 31:3715-3722 (2010); and Hwang et al., Biomaterials, 28:3560-3568 (2007). In some cases, the proline residues at position 6 and/or 7 of SEQ ID NO:13 are hydroxyproline residues. In some cases, the tyrosine residues at positions 5 and/or 9 of SEQ ID NO:13 are DOPA residues. In some cases, a mussel adhesive polypeptide that can be used as described herein can be a chimeric polypeptide that includes six decapeptide (AKPSYPPTYK; SEQ ID NO:13) repeats of MFP-1 at both the N- and C-termini of MFP-5. See, Lim, et al., Biomaterials, 31:3715-3722 (2010); and Hwang et al., Biomaterials, 28:3560-3568 (2007). In some cases, the proline residues at position 6 and/or 7 of SEQ ID NO:13 are hydroxyproline residues. In some cases, the tyrosine residues at positions 5 and/or 9 of SEQ ID NO:13 are DOPA residues. Mussel adhesive polypeptides can be extracted from any type of mussel or can be recombinantly produced using polypeptide expression techniques (e.g., heterologous expression techniques using bacterial cells, insect cells, or mammalian cells). Preparations of mussel adhesive polypeptides that are extracted from mussels are commercially available from Cell-Tek (Catalog No. 354240) and ACRO Biosystems (Catalog No. MAP-04012). In some cases, mussel adhesive polypeptides can be made as described elsewhere (e.g., Kitamura et al., J. Polymer Science: Part A: Polymer Chemistry, 37:729-736 (1991); Lim et al., Biomaterials, 31: 3715-3722 (2010); and Hwang et al., Biomaterials, 28:3560-3568 (2007)). In some cases, standard polypeptide synthesis techniques (e.g., liquid-phase polypeptide synthesis techniques or solid-phase polypeptide synthesis techniques) can be used to produce mussel adhesive polypeptides synthetically.

Other examples of adhesive molecules that can be used as described herein include polymers that include a plurality of DOPA residues. See, for example, the adhesive molecules in Table 1 that contain a plurality of DOPA residues. In some cases, such polymers can have one or more repeats of the consensus sequence XYX₄YX₃YX₃YYX₅YYYXYX₅YXYX₆YX₄YXYYX, where X is lysine, glycine, serine, histidine, or asparagine, and where Y refers to DOPA instead of tyrosine (SEQ ID NO:37). In cases in which serine is included in the polymer, a phosphoserine residue can be substituted for one or more of the serine residues. For example, in the adhesive molecule of any one of SEQ ID NOs:23-25, a phosphoserine residue can be substituted for one or more of the serine residues. In some cases, an adhesive molecule can be a polypeptide containing a random mixture of DOPA and lysine residues, a random mixture of DOPA, lysine, and glycine residues, or random mixture of DOPA and N5-(2-hydroxyethyl)-L-Glutamine. See, for example, Wang et al., Biomaterials, 28:3456-3468 (2007); and Anderson et al., Advanced Functional Materials, 20:4196-4205 (2010). An adhesive molecule also can be a polyamino acid containing catechols. See, for example, U.S. Pat. No. 6,506,577.

In some cases, an adhesion molecule can be a polypeptide that ranges in size from 10 to 1000 amino acids in length (e.g., 10 to 1000, 10 to 900, 10 to 800, 10 to 700, 10 to 600, 10 to 500, 10 to 400, 10 to 300, 10 to 200, 10 to 100, 20 to 750, 20 to 500, 20 to 350, 20 to 300, 20 to 250, 20 to 200, 20 to 150, 20 to 125, 20 to 100, 30 to 600, 30 to 550, 30 to 500, 30 to 450, 30 to 400, 30 to 350, 30 to 300, 30 to 250, 30 to 200, 30 to 150, 30 to 125, 30 to 100, 50 to 750, 50 to 700, 50 to 650, 50 to 600, 50 to 550, 50 to 500, 50 to 450, 50 to 400, 50 to 350, 50 to 300, 50 to 250, 50 to 200, 50 to 150, 50 to 125, or 50 to 100 amino acids in length). In some cases, an adhesion molecule can be a polypeptide that ranges in size from 5 to 10 amino acids in length.

Suitable polymers can have a peptidic or non-peptidic backbone, and can be synthesized by solid phase or solution phase synthesis. Such synthesis techniques allow a high percentage of DOPA (e.g., greater than 10%, greater than 15%, greater than 20%, greater than 30% of DOPA, or greater than 40% DOPA) to be incorporated into the polymers.

TABLE 1
Adhesive molecules containing
a plurality of DOPA residues.

| Adhesive Molecule, | SEQ |
|---|---|
| where Y refers to L-DOPA | ID |
| KYKGKGYKGGYKGKYYGKGKKYYYK | 21 |
| YKGKGKYKYGKKKGKYKGKGYKYYG | |
| KYKKKKYKKKYKKKYYKKKKKYYYK | 22 |
| YKKKKKYKYKKKKKKYKKKKYKYYK | |
| SYHGSGYHGGYKGKYYGKAKKYYYK | 23 |
| YKNSGKYKYLKKARKYHRKGYKYYG | |
| SYSSSSYSSSYKSKYYSKSKKYYYK | 24 |
| YKSSSKYKYSKKSSKYSSKSYKYYS | |
| GYSGKNYHGSYKGKYYHKHKKYYYK | 25 |
| YKLLHKYKYGKKGNKYGGKGYKYYH | |
| KYKYKYYKYYYKYKYYYKYKKYYYK | 26 |
| YKYKYKYKYYKKKYKYKYKYYKYYY | |
| KYKGKGGKGGYKGKGYGKGKKGYGK | 27 |
| YKGKGKGKYLKKKGKGKGKGYKGYG | |
| KYKKKKYKKKYKKKYYKKKKKYYYK | 28 |
| YKKKKKYKYKKKKKKYKKKKYKYYK | |
| KKGGYYKGKYKYGKKKKGGYYGGKY | 29 |
| YGKKKYGKYYYYYKGKYYGYKYGKK | |
| GKYYG | 30 |
| GKYGGGKYGG | 31 |
| KYYGKGKKYYYKYKG | 32 |
| KYYGKGKKYYYKG | 33 |
| KGYGKGKKYYYKG | 34 |
| KGKGKGKKYYYKG | 35 |
| KYGKYGKYKG | 36 |

An adhesive molecule can have a poly(acrylic acid) backbone with a plurality of DOPA residues. For example, an adhesive molecule can have a poly[butadiene-co-(maleic acid)] or poly[ethylene-co-(maleic acid)] backbone with a plurality of DOPA residues, and optionally a plurality of lysine residues, attached as side chains. See, for example, Laulicht et al., Macromol. Biosci., 12: 1555-1565 (2012). Such polymers containing a plurality of DOPA residues are soluble at basic pH values. The addition of lysine residues can increase the solubility at both acid and basic values. An adhesive molecule can have a polymethacrylate backbone with a plurality of DOPA residues, and optionally a plurality of lysine residues, incorporated. See, Kim et al., J. Porous Mater., 20:177-182 (2013). A polymethacrylate polymer containing a plurality of DOPA residues can be soluble at both acid and basic pH values.

In some cases, an adhesive molecule can be a DOPA containing polypeptide or a poly(dopamine) polymer. See, for example, Fuller et al., Biopolymers, 17:2939-2943 (1998); and Lee et al., Adv. Mater., 21:431-434 (2009). A poly(dopamine) polymer can be prepared, for example, by in situ polymerization.

In some cases, an adhesive molecule can be a polyethylene glycol terminated with DOPA. See, for example, Dalsin et al., J. Am. Chem. Soc., 125:4253-4258 (2003).

As described herein, an adhesive molecule provided herein can be used to adhere another agent (e.g., an interlocking metal, a dye, a polypeptide, a fluorescent molecule, a polymer, an antibiotic, or a therapeutic agent, a nucleic acid, a whitening particle, a rejuvenating particle, or a biological moiety) to hair, skin, and/or nails.

Examples of interlocking metals that can be adhered to or bound to an adhesive molecule provided herein include, without limitation, copper, iron oxides, zinc oxides, bismuth oxychloride or chromium oxides. In general, an adhesive molecule provided herein can adhere to or bind to hair, skin, and/or nails via one or more DOPA moieties and can adhere to or bind to an interlocking metal via, for example, one or more DOPA moieties. In some cases, an adhesive molecule provided herein can adhere to or bind to an interlocking metal via one or more thiol or maleimide groups.

Examples of dyes that can be adhered to or bound to an adhesive molecule provided herein include, without limitation, henna, guanine, prophyllite, ultramarine, and mica. In general, an adhesive molecule provided herein can adhere to or bind to hair, skin, and/or nails via one or more DOPA moieties and can adhere to or bind to a dye via, for example, one or more DOPA moieties. In some cases, an adhesive molecule provided herein can adhere to or bind to a dye via one or more thiol, hydroxide or maleimide groups.

Table 2 provides examples of agents that can be used to provide color to hair, skin, and/or nails.

TABLE 2
Examples of Agents Used to Provide Color

| Substrate | Color | Agent |
|---|---|---|
| Any | orange | Annatto extract |
| Any | purple | Dehydrated beets (beet powder) |
| Any | yellow | Canthaxanthin(3) |
| Any | brown | Caramel |
| Any | orange | β-Apo-8′-carotenal |
| Any | orange | β-Carotene |
| Any | red | Cochineal extract |
| Any | red | Carmine |
| Any | green | Sodium copper chlorophyllin(3) |
| Any | brown | Toasted partially defatted cooked |
| | | cottonseed flour |
| Any | brown | Ferrous gluconate |
| Any | brown | Ferrous lactate |
| Any | purple | Grape color extract(3) |
| Any | purple | Grape skin extract (enocianina) |
| Any | | Synthetic iron oxide(3) |
| Any | | Fruit juice(3) |
| Any | | Vegetable juice(3) |
| Any | orange | Carrot oil |
| Any | red | Paprika |
| Any | red | Paprika oleoresin |
| Any | | Mica-based pearlescent pigments(3) |
| Any | | Riboflavin |
| Any | orange | Saffron |
| Any | white | Titanium dioxide |
| Any | red | Tomato lycopene extract; tomato lycopene |
| | | concentrate(3) |
| Any | yellow | Turmeric |
| Any | yellow | Turmeric oleoresin |
| Any | blue | FD&C Blue No. 1 |
| Any | blue | FD&C Blue No. 2 |
| Any | green | FD&C Green No. 3 |
| Any | orange | Orange B(3) |
| Any | red | Citrus Red No. 2 |
| Any | red | FD&C Red No. 3 |
| Any | red | FD&C Red No. 40(3) |
| Any | yellow | FD&C Yellow No. 5 |
| Any | yellow | FD&C Yellow No. 6 |
| Any | aluminum | Alumina (dried aluminum hydroxide) |
| Any | white | Calcium carbonate |
| Any | | Potassium sodium copper chlorophyllin |
| | | (chlorophyllin-copper complex) |
| Any | | Dihydroxyacetone |
| Any | | Bismuth oxychloride |
| Any | | Synthetic iron oxide |
| Any | | Ferric ammonium ferrocyanide |
| Any | | Ferric ferrocyanide |
| Any | green | Chromium hydroxide green |
| Any | green | Chromium oxide greens |
| Any | | Guanine |
| Any | pearlescent | Mica-based pearlescent pigments (3) |
| Any | | Pyrophyllite |
| Any | | Mica |
| Any | | Talc |
| Any | aluminum | Aluminum powder |
| Any | bronze | Bronze powder |
| Any | copper | Copper powder |
| Any | zinc | Zinc oxide |
| Any | blue | D&C Blue No. 4 |
| Any | green | D&C Green No. 5 |
| Any | green | D&C Green No. 6 |
| Any | green | D&C Green No. 8 |
| Any | orange | D&C Orange No. 4 |
| Any | orange | D&C Orange No. 5 |
| Any | orange | D&C Orange No. 10 |
| Any | orange | D&C Orange No. 11 |
| Any | red | FD&C Red No. 4 |
| Any | red | D&C Red No. 6 |
| Any | red | D&C Red No. 7 |
| Any | red | D&C Red No. 17 |
| Any | red | D&C Red No. 21 |
| Any | red | D&C Red No. 22 |
| Any | red | D&C Red No. 27 |
| Any | red | D&C Red No. 28 |
| Any | red | D&C Red No. 30 |
| Any | red | D&C Red No. 31 |
| Any | red | D&C Red No. 33 |
| Any | red | D&C Red No. 34 |
| Any | red | D&C Red No. 36 |
| Any | red | D&C Red No. 39 |
| Any | violet | D&C Violet No. 2 |
| Any | yellow | D&C Yellow No. 7 |
| Any | yellow | Ext. D&C Yellow No. 7 |
| Any | Yellow | D&C Yellow No. 8 |
| Any | Yellow | D&C Yellow No. 10 |
| Any | Yellow | D&C Yellow No. 11 |
| Any | Orange | Annatto |
| any | Orange | β-Carotene |
| Hair | bismuth | Bismuth citrate(3) |
| Hair | green | Disodium EDTA-copper |
| Any | green | Potassium sodium copper chlorophyllin |
| | | (chlorophyllin copper-complex) |
| Any | zulene | Guaiazulene |
| Hair | brown | Henna(3) |
| Any | red | Iron oxides |
| Hair | lead | Lead acetate(3) |
| Nails | metallic | Silver (3) |
| Any | Blue | Ultramarines |
| Any | violet | Manganese violet |
| Any | yellow- | Luminescent zinc sulfide (3) |
| | green | |
| Nails and | Black | D&C Black No. 2 |
| skin | | |
| Nails and | Black | D&C Black No. 3 (3) |
| skin | | |
| Any | Brown | D&C Brown No. 1 |
| Any | Violet | Ext. D&C Violet No. 2 |

Examples of polypeptides that can be adhered to or bound to an adhesive molecule provided herein include, without limitation, keratin polypeptides, fluorescence emitting polypeptides, collagen polypeptides, and albumin polypeptides. In general, an adhesive molecule provided herein can adhere to or bind to hair, skin, and/or nails via one or more DOPA moieties and can adhere to or bind to a polypeptide via, for example, one or more DOPA moieties. In some cases, an adhesive molecule provided herein can adhere to or bind to a polypeptide via one or more thiol or maleimide groups. Any appropriate keratin polypeptide can be used as described herein.

Examples of keratin polypeptides that can be used as described herein include, without limitation, epidermal keratin (GenBank® Accession No. J00124; GI No. 186704), epithelial cell keratin (GenBank® Accession No. X13320.1; GI No. 1200071), and hair-specific keratin (GenBank® Accession No. AJ000263; GI No. 2695878).

Any appropriate fluorescence emitting polypeptide can be used as described herein. A fluorescence emitting polypeptides can emit fluorescence at a particular wavelength. In the case of BFP polypeptides, the BFP polypeptides can emit fluorescence in the range of about 440 nm to about 500 nm (e.g., between about 450 nm and about 500 nm, between about 460 nm and about 500 nm, between about 470 nm and about 500 nm, between about 480 nm and about 500 nm, between about 440 nm and about 490 nm, between about 440 nm and about 480 nm, between about 440 nm and about 470 nm, between about 440 nm and about 460 nm, between about 450 nm and about 490 nm, or between about 460 nm and about 480 nm). In some cases, a fluorescence emitting polypeptide that emits fluorescence at an emission wavelength of between about 420 nm and about 450 nm, between about 430 nm and about 450 nm, between about 440 nm and about 450 nm, between about 420 nm and about 440 nm, or between about 485 nm and about 505 nm can be used as described herein.

When the desire is to have hair, skin, and/or nails of a different color, a polypeptide that emits fluorescence in the red, green, or yellow spectrum can be used. Red fluorescence can have an emission wavelength between about 555 nm and about 655 nm (e.g., between about 565 nm and about 645 nm, between about 575 nm and about 635 nm, or between about 585 nm and about 625 nm). Green fluorescence can have an emission wavelength between about 500 nm and about 525 nm (e.g., between about 505 nm and about 520 nm or between about 510 nm and about 515 nm). Yellow fluorescence can have a wavelength between about 525 nm and about 555 nm (e.g., between about 530 nm and about 550 nm or 535 nm and about 545 nm). In some cases, a combination of different fluorescence emitting polypeptides can be used as described herein. For example, a combination of BFP polypeptides and red fluorescent protein (RFP) polypeptides can be applied to a person's hair, skin, and/or nails. In some cases, a combination of RFP polypeptides and green fluorescent protein (GFP) polypeptides can be applied to a person's hair, skin, and/or nails.

Any appropriate BFP polypeptide can be used as described herein. Examples of BFP polypeptides that can be used as described herein include, without limitation, EBFP (e.g., an EBFP having an emission max of 460 nm), fluorescent protein SBFP1 (GenBank® Accession No. ABM97856; GI No. 124264536), fluorescent protein SBFP2 (GenBank® Accession No. ABM97857, GI No. 124264538), EBFP2 (GenBank® Accession No. EF517318, GI No. 145666498), Azurite (Mena et al., Nature Biotechnology, 24:1569-1571 (2006)), mKalamal (GenBank® Accession No. EF517317, GI No. 145666496), zinc finger protein 383 (GenBank® Accession No. EDU39924.1, GI No. 187972425), SEQ ID NO:445 set forth in U.S. Pat. No. 7,166,424 (GenBank® Accession No. ABN30727.1; GI No. 125148618), soluble-modified blue fluorescent protein (smBFP) (GenBank® Accession No. U70497.1; GI No.1619752), polypeptides having the sequence set forth in GenBank® Accession No. CAE00365.1 (GI No. 32260521), polypeptides having the sequence set forth in GenBank® Accession No. CAE00361.1 (GI No. 32260509), polypeptides having the sequence set forth in GenBank® Accession No. CAE00361.1 (GI No. 32260509), ECFP polypeptides (GenBank® Accession No. AC048275.1; GI No. 226331138), Cerulean polypeptides (GenBank® Accession No. ADE48834.1; GI No. 293612838), Fluorescent Protein Cypet polypeptides (GenBank® Accession No. 3GEX_A; GI No. 290789997), MiCy polypeptides (GenBank® Accession No. ADE48830.1; GI No. 293612833), and mTFP1 fluorescent protein polypeptides (GenBank® Accession No. AC048263.1; GI No. 226320339). In some cases, a BFP polypeptide set forth in U.S. Patent Application Publication No. 2010/0062460 can be used as described herein.

Any appropriate RFP polypeptide and GFP polypeptide can be used as described herein. Examples of RFP polypeptides that can be used as described herein include, without limitation, soluble-modified red-shifted green fluorescent protein (smRSGFP) polypeptides (GenBank® Accession No. U70496.1; GI No.1619750), red fluorescent protein polypeptides having the sequence set forth in GenBank® Accession No. AAG16224.1 (GI No. 10304307); AB038175.1 (GI No.133753343); or AAU06852.1 (GI No. 51593130), Orange-Emitting GFP-Like Protein polypeptides (GenBank® Accession No. 2ZMW_D; GI No. 209870302), mOrange fluorescent protein polypeptides (GenBank® Accession No. AC048285.1; GI No. 226331152), NLS-dTomato polypeptides (GenBank® Accession No. ADC42843.1; GI No. 288188779), red fluorescent protein tdTomato polypeptides (GenBank® Accession No. ACQ43939.1; GI No. 228484713), DsRed polypeptides (GenBank® Accession No. BAE53441.1; GI No. 83016748), DsRed2 polypeptides (GenBank® Accession No. AAV73970.1; GI No. 56119204), DsRed-Express polypeptides (GenBank® Accession No. ACU30027.1; GI No. 255689290), DsRed-Monomer polypeptides (GenBank® Accession No. ACF35425.1; GI No. 194245628), monomeric orange-red fluorescent protein polypeptides (GenBank® Accession No. AAV52170.1; GI No. 55420625), monomeric orange-red fluorescent protein polypeptide (GenBank® Accession No. AAV52166.1; GI No. 55420617), mCherry polypeptides (GenBank® Accession No. ACY24904.1; GI No. 262089840), polypeptides having the amino acid sequence of SEQ ID NO:3 set forth in U.S. Pat. No. 7,393,923 (GenBank® Accession No. ACH06540.1; GI No. 197013979), and polypeptides having the amino acid sequence of SEQ ID NO:5 set forth in U.S. Pat. No. 7,393,923 (GenBank® Accession No. ACH06541.1; GI No. 197013980).

Examples of GFP polypeptides that can be used as described herein include, without limitation, soluble-modified green fluorescent protein (smGFP) polypeptides (GenBank® Accession No. U70495.1; GI No.1619748), modified green fluorescent protein GFP-ER (mfgp4-ER) polypeptides (GenBank® Accession No. U87625.1; GI No. 1842446), GFP polypeptides (GenBank® Accession No. ACJ06700.1, GI No. 210076685), enhanced GFP polypeptides (GenBank® Accession No. ACV20892.1; GI No. 256708579), turboGFP polypeptides (GenBank® Accession No. ADD23343.1; GI No. 290131407), VisGreen GFP polypeptides (GenBank® Accession No. ABR26680.1; GI No. 149393496), and Azami-Green polypeptides (GenBank® Accession No. BAD52001.1; GI No. 52839539).

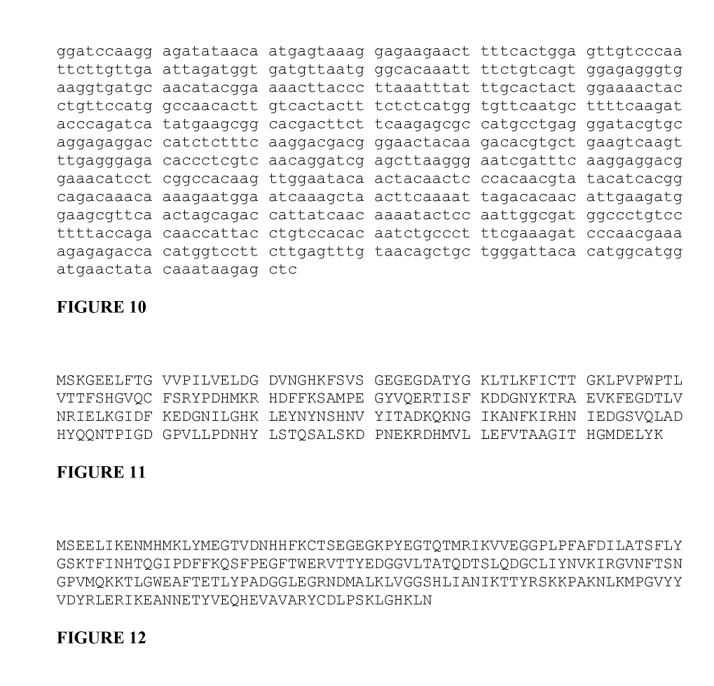

In some cases, a fluorescence emitting polypeptide such as those described by Subach et al. (Chem. Biol., 15:1116-1124 (2008)) can be used as described herein. See, also, GenBank® Accession No. 3M24_A (GI No. 296863586), GenBank® Accession No. 3M24_B (GI:296863587), GenBank® Accession No. 3M24_C (GI:296863588), and GenBank® Accession No. 3M24_D (GI:296863589). Additional examples of fluorescence emitting polypeptides that can be used as described herein include, without limitation, those described elsewhere (Alieva et al. PLoS ONE, 3(7):e2680 (2008) and Chudafov et al., Physiol. Rev., 90:1103-1163 (2010)). See, e.g., Table 1 of the Alieva et al. reference and FIGS. 5, 10, 12, and 14 of the Chudafov et al. reference. In some cases, a coral fluorescence emitting polypeptide can be used as described herein. In some cases, a fluorescence emitting polypeptide having the amino acid sequence set forth in FIG. 11 or 12 or having an amino acid sequence encoded by the sequence set forth in FIG. 10 can be used as described herein.

Any appropriate albumin polypeptide can be used as described herein. Examples of albumin polypeptides that can be used as described herein include, without limitation, human serum albumin (GenBank® Accession No. M12523; GI No. J04457), human albumin (GenBank® Accession No. EF649953.1; GI No. 152112963), and synthetic human albumin (GenBank® Accession No. DQ894588.2; GI No. 123995824).

Any appropriate method can be used to make a polypeptide (e.g., a keratin polypeptide, a fluorescence emitting polypeptide, or a non-fluorescent polypeptide).

For example, polypeptide expression techniques (e.g., heterologous expression techniques using bacterial cells, insect cells, or mammalian cells) can be used to make a polypeptide. In some cases, fluorescence emitting polypeptides such as BFP polypeptides can be made as described elsewhere (Yakhnin et al., Protein Expr. Purif., 14:382-386 (1998) and Jain et al., J. Chromatography A, 1035:83-86 (2004)). In some cases, standard polypeptide synthesis techniques (e.g., liquid-phase polypeptide synthesis techniques or solid-phase polypeptide synthesis techniques) can be used to produce polypeptides (e.g., a keratin polypeptide, a fluorescence emitting polypeptide, or a non-fluorescent polypeptide) synthetically.

In some cases, this document provides methods and materials for contacting hair, skin, and/or nails with an adhesive molecule and one or more interlocking metals and/or one or more dyes, and optionally one or more polypeptides (e.g., a keratin polypeptide) to provide the hair, skin, and/or nails with an improved appearance, an altered appearance, and/or a strengthened integrity.

In some cases, the adhesive molecule and other agent (e.g., interlocking metal, dye, polypeptide, nucleic acid, fluorescent moiety, antibiotic, or other drug) can be applied sequentially to hair, skin, and/or nails. For example, an adhesive molecule and one or more interlocking metals and/or one or more dyes can be applied sequentially, i.e., the adhesive molecule can be applied to hair, skin, and/or nails (under dry conditions or under wet conditions) and then the one or more interlocking metals and/or one or more dyes can be applied.

In some cases, the adhesive molecule and other agent (e.g., interlocking metal, dye, polypeptide, nucleic acid, fluorescent moiety, antibiotic, or other drug) can be applied together. For example, the adhesive molecule and one or more interlocking metals and/or one or more dyes can be attached to each other (e.g., conjugated to each other), and the complex can be applied to hair, skin, and/or nails.

A polypeptide can be covalently or non-covalently attached to an adhesive molecule such as a mussel adhesive polypeptide or polymer containing a plurality of DOPA residues. Any appropriate method can be used to covalently or non-covalently attach a polypeptide to an adhesive molecule (e.g., a polypeptide or polymer) having the ability to interact with or bind to hair, skin, and/or nails. For example, a polypeptide such as a keratin polypeptide, a collagen polypeptide, or an albumin polypeptide can be chemically conjugated to an adhesive molecule such as a mussel adhesive polypeptide or polymer via one or more coordinate covalent bonds, covalent bonds, disulfide bonds, high energy bonds, hydrogen bonds, ionic bonds, or peptide bonds. In some cases, a polypeptide can be chemically conjugated to an amine group present on a polypeptide having the ability to interact with or bind to hair, skin, and/or nails (e.g., a mussel adhesive polypeptide or other polypeptide with a plurality of DOPA residues). Such an amine group can be located at the N-terminus of the polypeptide, the C-terminus of the polypeptide, or in between the N- and C-termini of the polypeptide.

In some cases, the polypeptides to be conjugated can be activated prior to conjugation. For example, a polypeptide (e.g., an adhesive molecule, a keratin polypeptide, or a fluorescence emitting polypeptide) can be activated by incorporation of a reactive thiol group (e.g., by reaction with 2-iminothiolane such as a Traut's reagent, or reaction with a polyethylene glycol polymer containing a N-Succinimidyl 3-(2-pyridyldithio)-propionate (SPDP) moiety on one end and a N-hydroxysuccinimide ester on the other end, and cleavage of the SPDP moiety with a reducing agent such as dithiothreitol (DTT) to activate the thiol). For example, a mussel adhesive polypeptide can be thiolated by reaction with 2-iminothiolane (e.g., a Traut's reagent) as described elsewhere (McCall et al., Bioconjugate Chem., 1:222-226 (1990)). The reaction conditions can be varied to maximize the yield of molecules activated with one or two thiols to decrease the possibility that conjugation may interfere with binding to hair, skin, and/or nails. The degree of thiol incorporation with fluorescent or non-fluorescent polypeptides can be measured using a sensitive fluorescence assay as described elsewhere (Lacy et al., Analytical Biochemistry, 382:66-68 (2008)). Polypeptide conjugates can be directly synthesized with the DOPA peptides or added to the tail of a recombinant FP (e.g., by crosslinking).

An adhesive molecule or a polypeptide (e.g., a keratin polypeptide, a fluorescence emitting polypeptide, or a collagen polypeptide) can be substituted with one or more maleimide groups via reaction of the polypeptide's amines with a bifunctional reagent containing a maleimide group and a reactive N-hydroxysuccinimide ester (e.g., a polyethylene glycol polymer containing a maleimide group on one end and a reactive N-hydroxysuccinimide ester on the other). The maleimide substituted polypeptide can then be conjugated to the thiol groups of an adhesive molecule (e.g., an adhesive polypeptide) having the ability to interact with or bind to hair, skin, and/or nails. In some cases, a maleimide substituted adhesive molecule can be conjugated to the thio groups of a polypeptide. The degree to which the polypeptide (e.g., a keratin polypeptide, a fluorescence emitting polypeptide, or a collagen polypeptide) or adhesive molecule is substituted with maleimide groups can be varied as described elsewhere (Singh, Bioconjugate Chem., 5:348-351 (1994)).

Additional examples of conjugation methods that can be used to conjugate an adhesive molecule to a polypeptide include, without limitation, those described in elsewhere (e.g., Hermanson, G. T. Bioconjugate Techniques, Second Edition, 2008, Elsevier). See, e.g., Part I, Section 4 and Part II, Section 5.

In some cases, the color intensity or fluorescent signal that is obtained using the methods and materials provided herein can be enhanced by linking multiple interlocking metals, dyes, and/or fluorescence emitting polypeptides to a single adhesive molecule having the ability to interact with or bind to hair, skin, and/or nails.

In some cases, this amplification can be effectively accomplished by first preparing a polymer containing multiple interlocking metals, dyes, and/or fluorescence emitting polypeptides and then linking this polymer to an adhesive molecule (e.g., a mussel adhesive polypeptide) having the ability to interact with or bind to hair, skin, and/or nails. In some cases, a polymer can be formed to have multiple interlocking metals, dyes, and/or fluorescence emitting polypeptides linked to a polypeptide such as a casein polypeptide, and this polymer can be applied with the adhesive molecule. Examples of methods (e.g., polymerization methods) that can be used to form polymers containing multiple interlocking metals, dyes, and/or fluorescence emitting polypeptides include, without limitation, those described elsewhere (e.g., Hermanson, G. T. Bioconjugate Techniques, Second Edition, 2008, Elsevier). See, e.g., Part II, Section 25. See also U.S. Patent Publication No. 2010/0203533; U.S. Patent Publication No. 2013/0022555, including the sections describing conjugations to casein polypeptides; U.S. Pat. No. 4,657,853; U.S. Pat. No. 7,220,405, U.S. Pat. No. 8,263,056, and Hoshino et al., J. Biochem. 102:785-791 (1987).

Conjugating an adhesive molecule activated with multiple maleimide groups to a polypeptide containing multiple thiol groups can produce monomeric adhesive molecule—polypeptide conjugates as well as oligomers that contain different number of adhesive molecules and polypeptides. The molecular weight distribution of such covalently linked oligomers can be determined by gel electrophoresis under denaturing conditions (SDS-PAGE). The molecular weight distribution of the products depends on a number of factors such as the degree to which the adhesive molecule and polypeptide are activated, the pH of the conjugation reaction (e.g., about pH 5 to about pH 7, e.g., pH 5 to 6), and the ratio of adhesive molecule to polypeptide in the conjugation reaction.

In addition to the molecular weight distribution of oligomers that contain different number of adhesive molecules and polypeptides, such oligomers can associate via non-covalent interactions in a process called aggregation. The aggregation state of the oligomers can be determined by size exclusion chromatography analysis under native conditions. To minimize aggregation, factors such as the pH of the conjugation reaction, storage pH, and ionic strength can be modulated as well as the concentration of the adhesive molecule and polypeptide (e.g., fluorescence-emitting polypeptide) and the degree of activation. Typically, aggregation decreases when the pH of the conjugation reaction is between about pH 5 and about pH 6, and when the conjugation reaction is stored at a pH of about 5 to about pH 6.0. Aggregation also typically is minimized when the ionic strength is >5 mM, e.g., 50 mM. Lower concentration, e.g., <1 mg/mL, <0.5 mg/mL, <0.2 mg/mL, or <0.1 mg/mL of the adhesive molecule and polypeptide also can minimize aggregation.

Conjugation reactions between adhesive molecules and polypeptides (e.g., keratin polypeptides) also are performed such that the resulting conjugates are stable under conditions typically found on hair, skin, and/or nails (e.g., for a period of one to seven days, or for one or more weeks such as two, three, four, or more weeks) and do not result in oxidation or discoloration.

In some cases, a polypeptide can be produced as a fusion or chimeric polypeptide with a polypeptide having the ability to interact with or bind to hair, skin, and/or nails such that the fusion or chimeric polypeptide has the ability to interact with or bind to hair, skin, and/or nails. For example, heterologous polypeptide expression techniques or synthetic polypeptide synthesis techniques can be used to produce a single polypeptide chain having an amino acid sequence of a full-length polypeptide or fragment thereof and an amino acid sequence of an adhesive molecule having the ability to interact with or bind to hair, skin, and/or nails (e.g., an adhesive molecule such as a mussel adhesive polypeptide or a polymer containing a plurality of DOPA residues). For example, a chimeric polypeptide can include a full length keratin polypeptide or fragment thereof that is at least about 90 percent identical to a full length keratin polypeptide and an adhesive molecule (e.g., a full length mussel adhesive polypeptide or fragment thereof that is at least about 80 percent identical to the full length mussel adhesive polypeptide or a polymer containing a plurality of DOPA residues such as the polymers set forth in Table 1).

In some cases, the single polypeptide chain can have (a) an amino acid sequence of a polypeptide (e.g., a keratin polypeptide, a fluorescence emitting polypeptide, or a collagen polypeptide) followed by an amino acid sequence of a polypeptide having the ability to interact with or bind to hair, skin, and/or nails or (b) an amino acid sequence of a polypeptide having the ability to interact with or bind to hair, skin, and/or nails followed by an amino acid sequence of a polypeptide (e.g., a keratin polypeptide, a fluorescence emitting polypeptide, or a collagen polypeptide). In some cases, the single polypeptide chain can have one or more (e.g., one, two, three, four, or five) amino acid sequences with each encoding a polypeptide (e.g., a keratin polypeptide, a fluorescence emitting polypeptide, or a collagen polypeptide) and one or more (e.g., one, two, three, four, or five) amino acid sequences with each encoding a polypeptide having the ability to interact with or bind to hair, skin, and/or nails.

In some cases, a fusion or chimeric polypeptide provided herein can include other amino acid sequences (e.g., spacers or binding residues). For example, a fusion or chimeric polypeptide having an amino acid sequence of a polypeptide (e.g., a keratin polypeptide, a fluorescence emitting polypeptide, or a collagen polypeptide) and an amino acid sequence of a polypeptide having the ability to interact with or bind to hair, skin, and/or nails can include one or more additional amino acid residues such as glycine, lysine, alanine, arginine, asparagine, aspartic acid, cysteine, glutamine acid, glutamine, isoleucine, leucine, methionine, phenylalanine, threonine, tryptophan, proline, histidine, valine serine, tyrosine, ornithine, taurine, pyrolysine, or seleocysteine residues, or amino acid derivatives (e.g., 5-hydroxytryptophan, L-dihydroxyphenylalanine, or α-difluoromethylornithine). Such additional amino acid residues can be designed to be spacers (e.g., a string of five or more glycine residues) or can be designed to allow polypeptides or other molecules to be chemically conjugated to the fusion or chimeric polypeptide. For example, a fusion or chimeric polypeptide having an amino acid sequence of a polypeptide (e.g., a keratin polypeptide, a fluorescence emitting polypeptide, or a collagen polypeptide) and an amino acid sequence of a polypeptide having the ability to interact with or bind to hair, skin, and/or nails can include one, two, three, four, five, or more additional lysine residues such that one or more polypeptides having the ability to interact with or bind to hair, skin, and/or nails (e.g., mussel adhesive polypeptides) can be chemically conjugated to the fusion or chimeric polypeptide.

The methods described herein can include using the adhesive molecule and optionally another agent (e.g., a polypeptide) to apply coloring or whiting particles composed of, for example, hydroxyapatite, substituted hydroxyapatite, amorphous calcium phosphate, fluoride, calcium, magnesium, phosphate, iron, tin ions, titanium dioxide, bismuth oxychloride, iron oxides, chromium oxide, silver, aluminum, bronze, copper, manganese, zinc oxide, luminescent zinc sulfide, carmine, henna, guanine, pyrophyllite, mica, and any salt forms thereof (e.g., sodium hexametaphosphate, magnesium chloride, ferrous sulfate) to hair, skin, and/or nails to alter the color appearance of the hair, skin, and/or nails.

Coloring or whitening particles can be nanoparticles or microparticles, or aggregates of nanoparticles or microparticles, and can range in size from 1 nanometer (nm) to 100 micrometers (μm) in size such as 1 nm to 50 μm, 1 nm to 20 μm, 5 nm to 20 μm, 10 nm to 20 μm, 1 nm to 10 μm, 5 nm to 10 μm, 10 nm to 10 μm, 1 nm to 1 μm, 5 nm to 1 μm, 10 nm to 1 μm, 100 nm to 1 μm, 1 nm to 500 nm, 1 nm to 250 nm, 1 nm to 125 nm, 1 nm to 100 nm, 1 nm to 75 nm, 1 nm to 50 nm, 5 nm to 500 nm, 5 nm to 250 nm, 5 nm to 125 nm, 5 nm to 100 nm, 5 nm to 75 nm, 5 nm to 50 nm, 5 nm to 20 nm, 10 nm to 150 nm, 10 nm to 125 nm, 10 nm to 100 nm, 10 nm to 75 nm, 10 nm to 50 nm, or 50 nm to 150 nm, or 50 nm to 125 nm in size. Coloring or whitening particles can be composed of mica, hydroxyapatite, or titanium dioxide. Other useful coloring or whitening particles can be composed of bismuth oxychloride, iron oxides, chromium oxide, silver, aluminum, bronze, copper, manganese, luminescent zinc sulfide, silicon dioxide, zirconium silicate, calcium phosphate, or zinc oxide. See, e.g., Photochem. Photobiol. Sci., 9, 495-509 (2010); and U.S. Pat. No. 6,004,567.

In some cases, adhesive molecules and coloring or whitening particles (or other agents such as interlocking metals or dyes) can be applied sequentially. For example, the adhesive molecule can be applied to hair, skin, and/or nails and then the coloring or whitening particles (or other compound) can be applied. In some cases, an adhesive molecule and coloring or whitening particles (or other compound) can be applied at the same time. In some cases, an adhesive molecule, a polypeptide, and coloring or whitening particles can be applied sequentially. In some cases, an adhesive molecule, a polypeptide, and coloring or whitening particles can be applied at the same time. For example, coloring or whitening particles can be bound to a conjugate containing an adhesive molecule and a polypeptide, and the complex containing the coloring or whitening particles and conjugate can be applied to hair, skin, and/or nails. In some cases, using the coloring or whitening particles in combination with interlocking metals and/or dyes can help enhance the appearance of hair, skin, and/or nails.

In some cases in which titanium dioxide, iron oxide, chromium oxide, or other particles are used, the particles can be coated with one or more serum proteins such as albumin or immunoglobulin (e.g., by incubating the particles with serum) that bind non-specifically to titanium dioxide, and the coated particles can be applied in combination with an adhesive molecule (e.g., polymer containing a plurality of DOPA residues). In some cases, a serum protein can be activated and chemically conjugated to an adhesive molecule/polypeptide conjugate, and then the conjugate containing the serum protein, adhesive molecule, and polypeptide can be bound to coloring or whitening particles such as titanium dioxide, iron oxide, or chromium oxide particles.

A composition provided herein containing an adhesive molecule and one or more other molecules (e.g., a composition containing an adhesive molecule attached to an antibiotic or other therapeutic molecule such as conditioners (including sealants), colorants, fragrances, sunscreen agents, and the like along with other substances commonly used for hair, skin, or nails can be administered to a mammal's hair, skin, and/or nails. For example, a composition containing an adhesive molecule, an interlocking metal, and an antibiotic can be formulated as a gel. In some cases, the composition can include one or more pharmaceutical excipients.

Any appropriate method can be used to deliver a composition provided herein to hair, skin, and/or nails. For example, a composition provided herein can be incorporated into hair, skin, or nail care products or cosmetics such as shampoo, conditioner, hair gel, mousse, lotion, oils, sunscreens, soap, body wash, perfumes or nail polish. In some cases, the composition can include one or more pharmaceutical excipients. For example, a gel containing an adhesive molecule, an interlocking metal, and optionally a polypeptide can include one or more thickeners (e.g., mineral colloids or polyethylene glycol (PEG)), buffers, surfactants, and/or anti-bacterial agents (e.g., Triclosan or zinc chloride).

In some cases, an effective amount of a composition provided herein can be delivered to hair, skin, and/or nails to improve the appearance of the hair, skin, or nails, to alter the appearance of the hair, skin, or nails, and/or to strengthen the integrity of the hair, skin, or nails.

An effective amount of adhesive molecules, interlocking metals, dyes, polypeptides, coloring or whitening particles, combinations thereof, or a composition provided herein can be any amount that improves the appearance of hair, skin, or nails, alters the appearance of hair, skin, or nails, and/or strengthens the integrity of hair, skin, or nails without inducing significant toxicity. For example, a composition provided herein can be incorporated into hair, skin, or nail care products or cosmetics, such as shampoo, conditioner, hair gel, mousse, lotion, oils, sunscreens, soap, body wash, perfumes or nail polish in an amount that results in between about 0.0001 mg and about 100 mg (e.g., between about 0.001 mg and about 100 mg, between about 0.01 mg and about 100 mg, between about 0.1 mg and about 100 mg, between about 0.5 mg and about 100 mg, between about 0.5 mg and about 50 mg, between about 0.5 mg and about 25 mg, between about 1 mg and about 100 mg, between about 1 mg and about 50 mg, or between about 1 mg and about 25 mg) of colorant compound per gram of cosmetic or care product. It will be appreciated that the amount can be higher for certain formulations, e.g., conditioning agents with slow rates of release.

In some cases, a composition provided herein can be applied to hair, skin, or nails for a period of time. For example, a composition described herein can be applied (e.g., directly applied) to hair, skin, or nails and remain in contact with the hair, skin, or nails, without rinsing, for between 30 seconds and 10 minutes (e.g., between 30 seconds and 5 minutes, between 30 seconds and 2.5 minutes, between 30 seconds and two minutes, between 1 minute and 10 minutes, between 2 minutes and 10 minutes, or between one minute and 5 minutes). In some cases such as with a leave-in conditioning agent, a composition provided herein can be allowed to contact hair, skin, or nails for a period of time such that the composition saturates the hair, skin, or nails. The compositions described herein can be applied to hair, skin, or nails under wet or dry conditions.

In some cases, a person's hair, skin, or nails can be prepared prior to delivering a composition provided herein. For example, a person's hair, skin, or nails can be washed, brushed, or polished (e.g., polished with pumice) prior to delivering a composition provided herein. In some cases, the surface of hair, skin, or nails to be treated can be treated with one or more agents capable of exposing hair, skin or nail binding sites. For example, hair, skin, or nails to be treated with a composition provided herein can be contacted with EDTA, phosphoric acid, acetone, or pumice to expose binding sites present on the hair, skin, or nails.

In some cases, a two or more step process can be used to apply an adhesive molecule and other agents to hair, skin, or nails. For example, a composition containing an adhesive molecule (e.g., mussel adhesive polypeptide or polymer containing a plurality of DOPA residues) having the ability to interact with or bind to hair, skin, or nails can be delivered to the hair, skin, or nails to be treated as one step followed by a step of delivering, for example, an interlocking metal, a dye, and/or a polypeptide having the ability to interact with or bind to the adhesive molecule. In some case, these two steps can be performed at the same time using a single composition that contains the molecule separate from the polypeptide or using separate compositions where one composition contains the adhesive molecule and another composition contains one or more other agents.

In some cases, an assay can be performed to confirm that a composition provided herein or a component of a composition provided herein (e.g., a mussel adhesive polypeptide or polymer containing a plurality of DOPA residues) has binding affinity for hair, skin, or nails. For example, a material to be tested can be incubated with a hair, skin, or nail matrix, and the amount of material in solution after binding can be compared with the initial concentration to determine, by difference, the amount of bound material. See, e.g., Raj et al., J. Biol. Chem., 267:5968-5976 (1992). In the case of a polypeptide material, the polypeptide concentration in solution can be measured using a bicinchoninic acid assay and/or an ortho-phthalaldehyde amine assay. Binding constants can be determined using the Langmuir Model (Bouropoulos and Moradian-Oldak, Calcif. Tissue Int., 72:599-603 (2003)). Any appropriate method can be used to assess the affinity of a composition provided herein for hair, skin, or nails or a hair, skin, or nail matrix. For example, bound and unbound compositions can be quantified.

Any appropriate method can be used to assess a composition provided herein for the ability to alter the appearance of hair, skin, or nails. For example, visual inspection techniques can be used to determine whether or not a composition provided herein can alter the appearance of hair, skin, or nails. Such visual inspection techniques can include using shade guides for comparison as described elsewhere (Paravina et al., J. Esthet. Restor. Dent., 19:276-283 (2007)). In some cases, the ability of a composition provided herein to alter the appearance of hair, skin, or nails can be measured using reflectance spectrophotometry. In such cases, the hair, skin, or nails can be illuminated with a white light source and analyzed as to the amount of light absorbed at different wavelengths by reflectance spectrophotometry (colorimetry). These measurements can then be repeated with the UV light filtered from the light source. The difference in the reflectance values obtained with the inclusion and exclusion of UV light is the UV fluorescence spectrum of the hair, skin, or nail surface. In some cases, the ability of a composition described herein to bind interlocking metals to hair, skin, or nails can be observed, for example, using scanning electron microscopy, profilometry, or biopanning.

A composition provided herein can have a low risk of toxicity to the person using the composition, can contain one or more polypeptides of human origin, can contain one or more polypeptides naturally present in food or drink products, and/or can lack potentially toxic dyes.

This document also provides delivery structures, methods of making delivery structures, and methods for using delivery structures to deliver agents to hair, skin, or nails. For example, polymer thin films can be produced using three different polymers: polymethacrylic acid (PMA), poly[ethylene-co-(maleic anhydride) (PEMA), and poly[butadiene-co-(maleic anhydride) (PBMA). For example, poly-L-DOPA-PMA polymers can be synthesized using different concentrations of DOPA using 1-Ethyl-3-(3-dimethylaminopropyl) carbodiimide and N-hydroxysuccinimide dissolved in a PBS solution containing PMA solution. L-DOPA can be added to the polymer solution and the precipitated poly(L-DOPA) desiccated to produce the polymer.

Polymer thin films can be produced using synthesized Poly-L-DOPA-PEMA and Poly-L-DOPA-PBMA polymers. For example, poly [ethylene-co-(maleic anhydride)] and poly[butadiene-co-(maleic anhydride)], separately, can each be added DOPA in ethanol solution. The reaction can be heated extensively and cooled, and the precipitated polymers desiccated to produce the polymer (e.g., a catecholamine/lysine polymer mimetic on a methacrylate backbone).

To create thin films, the poly-L-DOPA solution can be layered on a tray and frozen in thin sheets. The frozen solution can be lyophilized, producing a film that retains its sheet structure. On one side of the film, agent solutions can be placed for delivery to hair, skin, or nails. For example, a layer of conditioning or therapeutic agents can be applied on one side of the film and placed on the surface of hair, skin and nails.

The invention will be further described in the following examples, which do not limit the scope of the invention described in the claims.

### EXAMPLES

### Example 1

### Method of Preparing Coloring Compound Using Interlocking Metals for Use in Coloring Hair

22.6 kDa mussel adhesive polypeptides (MAP-22) are activated for conjugation by thiolating the lysine residues using Traut's reagent (2-iminothiolane (IT)). The MAP-22 (0.4 mg/mL) are incubated with 1.8 mM or 5 mM IT at pH 8 for 40 minutes at room temperature in the presence of sodium borate to protect the DOPA residues of MAP-22 from oxidation. Two batches of MAP-22 are produced with different numbers of thiols attached, MAP-22-Hi-SH (5 mM IT) and MAP-22-Lo-SH (1.8 mM IT).

In this case, Cu is used as a coloring agent, but similar metals can be used instead of Cu, such as bismuth oxychloride, iron oxides, chromium oxide, silver, aluminum, bronze, copper, manganese, zinc oxide, or luminescent zinc sulfide. A series of small scale combination reactions are performed that contain the following:
Three reactions containing MAP-Lo-SH+Cu at MAP-22/Cu ratios of 3:1, 1:1, and 1:3.
Three reactions containing MAP-Hi-SH+Cu at MAP-22/Cu ratios of 3:1, 1:1, and 1:3.

The reaction products are applied to hair and allowed to incubate at room temperature for 20 minutes. After incubation, the hair sample is rinsed with water, and the sample is analyzed for effectiveness of coloration.

### Example 2

### Method of Preparing Coloring Compound Using Interlocking Metals for Use in Coloring Nails

MAP-22 are activated for conjugation by thiolating the lysine residues using Traut's reagent (2-iminothiolane (IT)). The MAP-22 (0.4 mg/mL) are incubated with 1.8 mM or 5 mM IT at pH 8 for 40 minutes at room temperature in the presence of sodium borate to protect the DOPA residues of MAP-22 from oxidation. Two batches of MAP-22 are produced with different numbers of thiols attached, MAP-22-Hi-SH (5 mM IT) and MAP-22-Lo-SH (1.8 mM IT).

In this case, Cu is used as a coloring agent, but similar metals can be used instead of Cu, such as bismuth oxychloride, iron oxides, chromium oxide, silver, aluminum, bronze, copper, manganese, zinc oxide, or luminescent zinc sulfide. A series of small scale combination reactions are performed that contain the following:
Three reactions containing MAP-Lo-SH+Cu at MAP-22/Cu ratios of 3:1, 1:1, and 1:3.
Three reactions containing MAP-Hi-SH+Cu at MAP-22/Cu ratios of 3:1, 1:1, and 1:3.

The reaction products are applied to nails and allowed to incubate at room temperature for 20 minutes. After incubation, the nail sample is rinsed with water, and sample is analyzed for effectiveness of coloration.

### Example 3

### Method of Preparing Coloring Compound Using Interlocking Metals for Use in Coloring Skin

MAP-22 are activated for conjugation by thiolating the lysine residues using Traut's reagent (2-iminothiolane (IT)). The MAP-22 (0.4 mg/mL) are incubated with 1.8 mM or 5 mM IT at pH 8 for 40 minutes at room temperature in the presence of sodium borate to protect the DOPA residues of MAP-22 from oxidation. Two batches of MAP-22 are produced with different numbers of thiols attached, MAP-22-Hi-SH (5 mM IT) and MAP-22-Lo-SH (1.8 mM IT).

In this case, Cu is used as a coloring agent, but similar metals can be used instead of Cu, such as bismuth oxychloride, iron oxides, chromium oxide, silver, aluminum, bronze, copper, manganese, zinc oxide, or luminescent zinc sulfide. A series of small scale combination reactions are performed that contain the following:
Three reactions containing MAP-Lo-SH+Cu at MAP-22/Cu ratios of 3:1, 1:1, and 1:3.
Three reactions containing MAP-Hi-SH+Cu at MAP-22/Cu ratios of 3:1, 1:1, and 1:3.

The reaction products are applied to skin and allowed to incubate at room temperature for 20 minutes. After incubation, the skin sample is rinsed with water, and sample is analyzed for effectiveness of coloration.

### Example 4

### Method of Preparing Coloring Compound Using Minerals and Dyes for Use in Coloring Hair

MAP-22 are activated for conjugation by thiolating the lysine residues using Traut's reagent (2-iminothiolane (IT)). The MAP-22 (0.4 mg/mL) are incubated with 1.8 mM or 5 mM IT at pH 8 for 40 minutes at room temperature in the presence of sodium borate to protect the DOPA residues of MAP-22 from oxidation. Two batches of MAP-22 are produced with different numbers of thiols attached, MAP-22-Hi-SH (5 mM IT) and MAP-22-Lo-SH (1.8 mM IT).

In this case, carmine is used as a coloring agent, but similar minerals and dyes can be used such as henna, guanine, pyrophyllite, or mica. A series of small scale combination reactions are performed that contain the following:
Three reactions containing MAP-Lo-SH+Carmine at MAP-22/Carmine ratios of 3:1, 1:1, and 1:3.
Three reactions containing MAP-Hi-SH+Carmine at MAP-22/ Carmine ratios of 3:1, 1:1, and 1:3.

The reaction products are applied to hair and allowed to incubate at room temperature for 20 minutes. After incubation, the hair sample is rinsed with water and analyzed for effectiveness of coloration.

### Example 5

### Method of Preparing Coloring Compound Using Minerals and Dyes for Use in Coloring Nails

MAP-22 are activated for conjugation by thiolating the lysine residues using Traut's reagent (2-iminothiolane (IT)). The MAP-22 (0.4 mg/mL) are incubated with 1.8 mM or 5 mM IT at pH 8 for 40 minutes at room temperature in the presence of sodium borate to protect the DOPA residues of MAP-22 from oxidation. Two batches of MAP-22 are produced with different numbers of thiols attached, MAP-22-Hi-SH (5 mM IT) and MAP-22-Lo-SH (1.8 mM IT).

In this case, carmine is used as a coloring agent, but similar minerals and dyes can be used such as henna, guanine, pyrophyllite, or mica. A series of small scale combination reactions are performed that contain the following:
Three reactions containing MAP-Lo-SH+Carmine at MAP-22/Carmine ratios of 3:1, 1:1, and 1:3.
Three reactions containing MAP-Hi-SH+Carmine at MAP-22/Carmine ratios of 3:1, 1:1, and 1:3.

The reaction products are applied to nails and allowed to incubate at room temperature for 20 minutes. After incubation, the nail sample is rinsed with water and analyzed for effectiveness of coloration.

### Example 6

### Method of Preparing Coloring Compound Using Minerals and Dyes for Use in Coloring Skin

MAP-22 are activated for conjugation by thiolating the lysine residues using Traut's reagent (2-iminothiolane (IT)). The MAP-22 (0.4 mg/mL) are incubated with 1.8 mM or 5 mM IT at pH 8 for 40 minutes at room temperature in the presence of sodium borate to protect the DOPA residues of MAP-22 from oxidation. Two batches of MAP-22 are produced with different numbers of thiols attached, MAP-22-Hi-SH (5 mM IT) and MAP-22-Lo-SH (1.8 mM IT).

In this case, carmine is used as a coloring agent, but similar minerals and dyes can be used, such as henna, guanine, pyrophyllite, or mica. A series of small scale combination reactions are performed that contain the following:
Three reactions containing MAP-Lo-SH+Carmine at MAP-22/Carmine ratios of 3:1, 1:1, and 1:3.
Three reactions containing MAP-Hi-SH+Carmine at MAP-22/ Carmine ratios of 3:1, 1:1, and 1:3.

The reaction products are applied to skin and allowed to incubate at room temperature for 20 minutes. After incubation, the skin sample is rinsed with water and analyzed for effectiveness of coloration.

### Example 7

### Method of Preparing Coloring Compound Using Interlocking Metals and Keratin as a Scaffolding Protein for Use in Coloring Hair

Keratin, used as a scaffold polymer to increase the size of the molecule, is prepared and activated as follows. The scaffold polymer keratin is split into two batches: one batch is activated with Trauts reagents (2-Iminothiolane or 2-IT) to add thiol groups to the scaffold polymer, and the second batch is activated with MAL-dPEG4-NHS ester to add maleimide groups to the scaffold polymer keratin.

The activation of scaffold polymer keratin with Trauts reagent is done in 50 mM borate buffer pH 8.0 containing 2 mM EDTA and 0.05% Tween-20. The final concentration of iminothiolane is 45 mM. The scaffold polymer keratin is maintained at 1 mg/mL. The reaction is incubated at room temperature for 40 minutes. Unreacted IT is removed from the reaction by gel filtration in 50 mM phosphate buffer, 2 mM EDTA, and 0.05% Tween-20 pH 7.0. In the maleimide reaction, scaffold polymer keratin is at a concentration of 1 mg/mL, and MAL-dPEG4-NHS ester is added to yield a final concentration of 3 mg/mL in a buffer of 50 mM phosphate, 2 mM EDTA, and 0.05% Tween-20. The reaction is incubated for 40 minutes at room temperature. To purify the resulting reaction of conjugated materials, unreacted MAL-dPEG4-NHS ester is separated from scaffold polymer keratin by gel filtration chromatography on a Sephadex G25 column (GE Healthcare) in 5 mM MES, 2 mM EDTA, and 0.05% Tween-20, pH 6.

The two activated scaffold polymer keratin species are then reacted together to form a poly-scaffold polymer. Activated scaffold polymer keratin species are combined at a 1:3 ratio of scaffold polymer keratin-maleimide and scaffold polymer keratin-SH and incubated for 1 hour at room temperature in 50 mM phosphate buffer, pH 7.0, 2 mM EDTA, and 0.05% Tween-20. The reaction is quenched with NMM. The sample is concentrated and applied to a size exclusion column (Superdex 200, GE Healthcare) to separate poly-scaffold polymer keratin of different sizes and to remove unreacted scaffold polymer keratin.

In order to react poly-scaffold polymer keratin with MAP-22-SH, a poly-scaffold polymer keratin fraction with a molecular weight of more than 220 kDa is used. Polymeric scaffold polymer keratin is activated by reaction with 0.33 mg/mL MAL-dPEG4-NHS ester in a 50 mM phosphate buffer containing 2 mM EDTA and 0.05% Tween 20 at pH 7. After unreacted reagent is removed by gel filtration, it is reacted with MAP-22-SH (activated with 5 mM iminothiolane) at different MAP-22/Poly-keratin ratios (MAP-22:Keratin; 4:1; 2:1; 1:1; 1:2; 1:4).

Under these conditions, there is sufficient material to coat the polymeric scaffolding protein keratin and further polymerization or aggregation followed by precipitation may not occur.

In this case, Cu is used as a coloring agent, but similar metals can be used instead of Cu, such as bismuth oxychloride, iron oxides, chromium oxide, silver, aluminum, bronze, copper, manganese, zinc oxide, or luminescent zinc sulfide. A series of small scale combination reactions are performed that contain the following:
Three reactions containing MAP-22:Kerati +Cu at MAP-22:Keratin/Cu ratios of 3:1, 1:1, and 1:3.

This compound is applied to hair and allowed to incubate at room temperature for 20 minutes. After incubation, the hair sample is rinsed with water and analyzed for effectiveness of coloration.

### Example 8

### Method of Preparing Coloring Compound Using Interlocking Metals and Keratin as a Scaffolding Protein for Use in Coloring Nails

Keratin, used as a scaffold polymer to increase the size of the molecule, is prepared and activated as follows. The scaffold polymer keratin is split into two batches: one batch is activated with Trauts reagents (2-Iminothiolane or 2-IT) to add thiol groups to the scaffold polymer, and the second batch is activated with MAL-dPEG4-NHS ester to add maleimide groups to the scaffold polymer keratin.

The activation of scaffold polymer keratin with Trauts reagent is done in 50 mM borate buffer pH 8.0 containing 2 mM EDTA and 0.05% Tween-20. The final concentration of iminothiolane is 45 mM. The scaffold polymer keratin is maintained at 1 mg/mL. The reaction is incubated at room temperature for 40 minutes. Unreacted IT is removed from the reaction by gel filtration in 50 mM phosphate buffer, 2 mM EDTA, and 0.05% Tween-20 pH 7.0. In the maleimide reaction, scaffold polymer keratin is at a concentration of 1 mg/mL, and MAL-dPEG4-NHS ester is added to yield a final concentration of 3 mg/mL in a buffer of 50 mM phosphate, 2 mM EDTA, and 0.05% Tween-20. The reaction is incubated for 40 minutes at room temperature. To purify the resulting reaction of conjugated materials, unreacted MAL-dPEG4-NHS ester is separated from scaffold polymer keratin by gel filtration chromatography on a Sephadex G25 column (GE Healthcare) in 5 mM MES, 2 mM EDTA, and 0.05% Tween-20, pH 6.

The two activated scaffold polymer keratin species are then reacted together to form a poly-scaffold polymer. Activated scaffold polymer keratin species are combined at a 1:3 ratio of scaffold polymer keratin-maleimide and scaffold polymer keratin-SH and incubated for 1 hour at room temperature in 50 mM phosphate buffer, pH 7.0, 2 mM EDTA, and 0.05% Tween-20. The reaction is quenched with NMM. The sample is concentrated and applied to a size exclusion column (Superdex 200, GE Healthcare) to separate poly-scaffold polymer keratin of different sizes and to remove unreacted scaffold polymer keratin.

In order to react poly-scaffold polymer keratin with MAP-22-SH, a poly-scaffold polymer keratin fraction with a molecular weight of more than 220 kDa is used. Polymeric scaffold polymer keratin is activated by reaction with 0.33 mg/mL MAL-dPEG4-NHS ester in a 50 mM phosphate buffer containing 2 mM EDTA and 0.05% Tween 20 at pH 7. After unreacted reagent is removed by gel filtration, it is reacted with MAP-22-SH (activated with 5 mM iminothiolane) at different MAP-22/Poly-keratin ratios (MAP-22:Keratin; 4:1; 2:1; 1:1; 1:2; 1:4).

Under these conditions, there is sufficient material to coat the polymeric scaffolding protein keratin and further polymerization or aggregation followed by precipitation may not occur.

In this case, Cu is used as a coloring agent, but similar metals can be used instead of Cu, such as bismuth oxychloride, iron oxides, chromium oxide, silver, aluminum, bronze, copper, manganese, zinc oxide, or luminescent zinc sulfide. A series of small scale combination reactions are performed that contain the following:
Three reactions containing MAP-22:Keratin+Cu at MAP-22:Keratin/Cu ratios of 3:1, 1:1, and 1:3.

This compound is applied to nails and allowed to incubate at room temperature for 20 minutes. After incubation, the nail sample is rinsed with water and analyzed for effectiveness of coloration.

### Example 9

### Method of Preparing Coloring Compound Using Interlocking Metals and Albumin as a Scaffolding Protein for Use in Coloring Skin

Albumin, used as a scaffold polymer to increase the size of the molecule, is prepared and activated as follows. The scaffold polymer albumin is split into two batches: one batch is activated with Trauts reagents (2-Iminothiolane or 2-IT) to add thiol groups to the scaffold polymer, and the second batch is activated with MAL-dPEG4-NHS ester to add maleimide groups to the scaffold polymer albumin. The activation of scaffold polymer albumin with Trauts reagent is done in 50 mM borate buffer pH 8.0 containing 2 mM EDTA and 0.05% Tween-20. The final concentration of iminothiolane is 45 mM. The scaffold polymer albumin is maintained at 1 mg/mL. The reaction is incubated at room temperature for 40 minutes. Unreacted IT is removed from the reaction by gel filtration in 50 mM phosphate buffer, 2 mM EDTA, and 0.05% Tween-20 pH 7.0. In the maleimide reaction, scaffold polymer albumin is at a concentration of 1 mg/mL, and MAL-dPEG4-NHS ester is added to yield a final concentration of 3 mg/mL in a buffer of 50 mM phosphate, 2 mM EDTA, and 0.05% Tween-20. The reaction is incubated for 40 minutes at room temperature. To purify the resulting reaction of conjugated materials, unreacted MAL-dPEG4-NHS ester is separated from scaffold polymer albumin by gel filtration chromatography on a Sephadex G25 column (GE Healthcare) in 5 mM MES, 2 mM EDTA, and 0.05% Tween-20, pH 6.

The two activated scaffold polymer albumin species are then reacted together to form a poly-scaffold polymer. Activated scaffold polymer albumin species are combined at a 1:3 ratio of scaffold polymer albumin-maleimide and scaffold polymer albumin-SH and incubated for 1 hour at room temperature in 50 mM phosphate buffer, pH 7.0, 2 mM EDTA, and 0.05% Tween-20. The reaction is quenched with NMM. The sample is concentrated and applied to a size exclusion column (Superdex 200, GE Healthcare) to separate poly-scaffold polymer albumin of different sizes and to remove unreacted scaffold polymer albumin.

In order to react poly-scaffold polymer albumin with MAP-22-SH, a poly-scaffold polymer albumin fraction with a molecular weight of more than 220 kDa is used. Polymeric scaffold polymer albumin is activated by reaction with 0.33 mg/mL MAL-dPEG4-NHS ester in a 50 mM phosphate buffer containing 2 mM EDTA and 0.05% Tween 20 at pH 7. After unreacted reagent is removed by gel filtration, it is reacted with MAP-22-SH (activated with 5 mM iminothiolane) at different MAP-22/Poly-albumin ratios (MAP-22:Albumin; 4:1; 2:1; 1:1; 1:2; 1:4).

Under these conditions, there is sufficient material to coat the polymeric scaffolding protein albumin and further polymerization or aggregation followed by precipitation may not occur.

In this case, Cu is used as a coloring agent, but similar metals can be used instead of Cu, such as bismuth oxychloride, iron oxides, chromium oxide, silver, aluminum, bronze, copper, manganese, zinc oxide, or luminescent zinc sulfide. A series of small scale combination reactions are performed that contain the following:
Three reactions containing MAP-22:Abumin+Cu at MAP-22:Albumin/Cu ratios of 3:1, 1:1, and 1:3.

This compound is applied to skin and allowed to incubate at room temperature for 20 minutes. After incubation, the skin sample is rinsed with water and analyzed for effectiveness of coloration.

### Example 10

### Method of Preparing Coloring Compound Using Minerals/Dyes and Keratin as a Scaffolding Protein for Use in Coloring Hair

Keratin, used as a scaffold polymer to increase the size of the molecule, is prepared and activated as follows. The scaffold polymer keratin is split into two batches: one batch is activated with Trauts reagents (2-Iminothiolane or 2-IT) to add thiol groups to the scaffold polymer, and the second batch is activated with MAL-dPEG4-NHS ester to add maleimide groups to the scaffold polymer keratin.

The activation of scaffold polymer keratin with Trauts reagent is done in 50 mM borate buffer pH 8.0 containing 2 mM EDTA and 0.05% Tween-20. The final concentration of iminothiolane is 45 mM. The scaffold polymer keratin is maintained at 1 mg/mL. The reaction is incubated at room temperature for 40 minutes. Unreacted IT is removed from the reaction by gel filtration in 50 mM phosphate buffer, 2 mM EDTA, and 0.05% Tween-20 pH 7.0. In the maleimide reaction, scaffold polymer keratin is at a concentration of 1 mg/mL, and MAL-dPEG4-NHS ester is added to yield a final concentration of 3 mg/mL in a buffer of 50 mM phosphate, 2 mM EDTA, and 0.05% Tween-20. The reaction is incubated for 40 minutes at room temperature. To purify the resulting reaction of conjugated materials, unreacted MAL-dPEG4-NHS ester is separated from scaffold polymer keratin by gel filtration chromatography on a Sephadex G25 column (GE Healthcare) in 5 mM MES, 2 mM EDTA, and 0.05% Tween-20, pH 6.

The two activated scaffold polymer keratin species are then reacted together to form a poly-scaffold polymer. Activated scaffold polymer keratin species are combined at a 1:3 ratio of scaffold polymer keratin-maleimide and scaffold polymer keratin-SH and incubated for 1 hour at room temperature in 50 mM phosphate buffer, pH 7.0, 2 mM EDTA, and 0.05% Tween-20. The reaction is quenched with NMM. The sample is concentrated and applied to a size exclusion column (Superdex 200, GE Healthcare) to separate poly-scaffold polymer keratin of different sizes and to remove unreacted scaffold polymer keratin.

In order to react poly-scaffold polymer keratin with MAP-22-SH, a poly-scaffold polymer keratin fraction with a molecular weight of more than 220 kDa is used. Polymeric scaffold polymer keratin is activated by reaction with 0.33 mg/mL MAL-dPEG4-NHS ester in a 50 mM phosphate buffer containing 2 mM EDTA and 0.05% Tween 20 at pH 7. After unreacted reagent is removed by gel filtration, it is reacted with MAP-22-SH (activated with 5 mM iminothiolane) at different MAP-22/Poly-keratin ratios (MAP-22:Keratin; 4:1; 2:1; 1:1; 1:2; 1:4).

Under these conditions, there is sufficient material to coat the polymeric scaffolding protein keratin and further polymerization or aggregation followed by precipitation may not occur.

In this case, carmine is used as a coloring agent, but similar minerals and dyes can be used, such as henna, guanine, pyrophyllite, or mica. A series of small scale combination reactions are performed that contain the following:
Three reactions containing MAP-22:Keratin+Carmine at MAP-22/Carmine ratios of 3:1, 1:1, and 1:3.

The reaction products are applied to hair and allowed to incubate at room temperature for 20 minutes. After incubation, the hair sample is rinsed with water and analyzed for effectiveness of coloration.

### Example 11

### Method of Preparing Coloring Compound Using Minerals/Dyes and Keratin as a Scaffolding Protein for Use in Coloring Nails

Keratin, used as a scaffold polymer to increase the size of the molecule, is prepared and activated as follows. The scaffold polymer keratin is split into two batches: one batch is activated with Trauts reagents (2-Iminothiolane or 2-IT) to add thiol groups to the scaffold polymer, and the second batch is activated with MAL-dPEG4-NHS ester to add maleimide groups to the scaffold polymer keratin.

The activation of scaffold polymer keratin with Trauts reagent is done in 50 mM borate buffer pH 8.0 containing 2 mM EDTA and 0.05% Tween-20. The final concentration of iminothiolane is 45 mM. The scaffold polymer keratin is maintained at 1 mg/mL. The reaction is incubated at room temperature for 40 minutes. Unreacted IT is removed from the reaction by gel filtration in 50 mM phosphate buffer, 2 mM EDTA, and 0.05% Tween-20 pH 7.0. In the maleimide reaction, scaffold polymer keratin is at a concentration of 1 mg/mL, and MAL-dPEG4-NHS ester is added to yield a final concentration of 3 mg/mL in a buffer of 50 mM phosphate, 2 mM EDTA, and 0.05% Tween-20. The reaction is incubated for 40 minutes at room temperature. To purify the resulting reaction of conjugated materials, unreacted MAL-dPEG4-NHS ester is separated from scaffold polymer keratin by gel filtration chromatography on a Sephadex G25 column (GE Healthcare) in 5 mM MES, 2 mM EDTA, and 0.05% Tween-20, pH 6.

The two activated scaffold polymer keratin species are then reacted together to form a poly-scaffold polymer. Activated scaffold polymer keratin species are combined at a 1:3 ratio of scaffold polymer keratin-maleimide and scaffold polymer keratin-SH and incubated for 1 hour at room temperature in 50 mM phosphate buffer, pH 7.0, 2 mM EDTA, and 0.05% Tween-20. The reaction is quenched with NMM. The sample is concentrated and applied to a size exclusion column (Superdex 200, GE Healthcare) to separate poly-scaffold polymer keratin of different sizes and to remove unreacted scaffold polymer keratin.

In order to react poly-scaffold polymer keratin with MAP-22-SH, a poly-scaffold polymer keratin fraction with a molecular weight of more than 220 kDa is used. Polymeric scaffold polymer keratin is activated by reaction with 0.33 mg/mL MAL-dPEG4-NHS ester in a 50 mM phosphate buffer containing 2 mM EDTA and 0.05% Tween 20 at pH 7. After unreacted reagent is removed by gel filtration, it is reacted with MAP-22-SH (activated with 5 mM iminothiolane) at different MAP-22/Poly-keratin ratios (MAP-22:Keratin; 4:1; 2:1; 1:1; 1:2; 1:4).

Under these conditions, there is sufficient material to coat the polymeric scaffolding protein keratin and further polymerization or aggregation followed by precipitation may not occur.

In this case, carmine is used as a coloring agent, but similar minerals and dyes can be used, such as henna, guanine, pyrophyllite, or mica. A series of small scale combination reactions are performed that contain the following:
Three reactions containing MAP-22:Keratin+Carmine at MAP-22/Carmine ratios of 3:1, 1:1, and 1:3.

The reaction products are applied to nails and allowed to incubate at room temperature for 20 minutes. After incubation, the nail sample is rinsed with water and analyzed for effectiveness of coloration.

### Example 12

### Method of Preparing Coloring Compound Using Minerals/Dyes and Albumin as a Scaffolding Protein for Use in Coloring Skin

Albumin, used as a scaffold polymer to increase the size of the molecule, is prepared and activated as follows. The scaffold polymer albumin is split into two batches: one batch is activated with Trauts reagents (2-Iminothiolane or 2-IT) to add thiol groups to the scaffold polymer, and the second batch is activated with MAL-dPEG4-NHS ester to add maleimide groups to the scaffold polymer albumin.

The activation of scaffold polymer albumin with Trauts reagent is done in 50 mM borate buffer pH 8.0 containing 2 mM EDTA and 0.05% Tween-20. The final concentration of iminothiolane is 45 mM. The scaffold polymer albumin is maintained at 1 mg/mL. The reaction is incubated at room temperature for 40 minutes. Unreacted IT is removed from the reaction by gel filtration in 50 mM phosphate buffer, 2 mM EDTA, and 0.05% Tween-20 pH 7.0. In the maleimide reaction, scaffold polymer albumin is at a concentration of 1 mg/mL, and MAL-dPEG4-NHS ester is added to yield a final concentration of 3 mg/mL in a buffer of 50 mM phosphate, 2 mM EDTA, and 0.05% Tween-20. The reaction is incubated for 40 minutes at room temperature. To purify the resulting reaction of conjugated materials, unreacted MAL-dPEG4-NHS ester is separated from scaffold polymer albumin by gel filtration chromatography on a Sephadex G25 column (GE Healthcare) in 5 mM MES, 2 mM EDTA, and 0.05% Tween-20, pH 6.

The two activated scaffold polymer albumin species are then reacted together to form a poly-scaffold polymer. Activated scaffold polymer albumin species are combined at a 1:3 ratio of scaffold polymer albumin-maleimide and scaffold polymer albumin-SH and incubated for 1 hour at room temperature in 50 mM phosphate buffer, pH 7.0, 2 mM EDTA, and 0.05% Tween-20. The reaction is quenched with NMM. The sample is concentrated and applied to a size exclusion column (Superdex 200, GE Healthcare) to separate poly-scaffold polymer albumin of different sizes and to remove unreacted scaffold polymer albumin.

In order to react poly-scaffold polymer albumin with MAP-22-SH, a poly-scaffold polymer albumin fraction with a molecular weight of more than 220 kDa is used. Polymeric scaffold polymer albumin is activated by reaction with 0.33 mg/mL MAL-dPEG4-NHS ester in a 50 mM phosphate buffer containing 2 mM EDTA and 0.05% Tween 20 at pH 7. After unreacted reagent is removed by gel filtration, it is reacted with MAP-22-SH (activated with 5 mM iminothiolane) at different MAP-22/Poly-albumin ratios (MAP-22:Albumin; 4:1; 2:1; 1:1; 1:2; 1:4).

Under these conditions, there is sufficient material to coat the polymeric scaffolding protein albumin and further polymerization or aggregation followed by precipitation may not occur.

In this case, carmine is used as a coloring agent, but similar minerals and dyes can be used, such as henna, guanine, pyrophyllite, or mica. A series of small scale combination reactions are performed that contain the following:
Three reactions containing MAP-22:Albumin+Carmine at MAP-22/Carmine ratios of 3:1, 1:1, and 1:3.

The reaction products are applied to skin and allowed to incubate at room temperature for 20 minutes. After incubation, the skin sample is rinsed with water and analyzed for effectiveness of coloration.

### Example 13

### Method of Preparing Thin Polymer Adhesive Films for Use as a Delivery Structure

Poly DOPA was synthesized using three different polymers: polymethacrylic acid (PMA), poly[ethylene-co-(maleic anhydride) (PEMA), and poly[butadiene-co-(maleic anhydride) (PBMA).

Poly-L-DOPA-PMA polymers were synthesized using different concentrations of DOPA. 1-Ethyl-3-(3-dimethylaminopropyl) carbodiimide (EDC; 0.5 g) and N-hydroxysuccinimide (NHS; 0.5 g) were dissolved in a PBS solution containing 2.5 g of PMA (MW 9.5 kDa, Sigma) solution. After the solution was mixed for 4 hours at room temperature, L-DOPA (0 g for PMA1, 0.25 g for PMA2, and 0.75 g for PMA3) was added to the respective polymer solutions (Table 3). The reaction volumes were adjusted to 10 mL with PBS, and the reactions were stirred for about 2 hours. The precipitated reaction products were then washed well with ethanol, the precipitated poly(L-DOPA) was dried, and the product stored in vacuum desiccator.

Poly-L-DOPA-PEMA and Poly-L-DOPA-PBMA polymers were synthesized as follows. In a round bottom flask attached with a cold water condenser, 0.5 g of each polymer, poly [ethylene-co- (maleic anhydride)] 1:1 (PEMA, MW, 400 kDa, Polysciences) and poly[butadiene-co-(maleic anhydride)] 1:1 (PBMA, MW 10-15 kDa, Polysciences), was added to 0.65 g DOPA in 50 mL ethanol. The reactions were refluxed at 70-90° C. on hot plates in a water or oil bath for 12 hours with constant stirring. After 12 hours, the reactions were cooled to room temperature, and the precipitated polymers were dried and stored in a vacuum desiccator (Table 3).

TABLE 3
Synthesis of poly-L-DOPA with PMA, PEMA, and PBMA.

| | Molecular | | | | |
|---|---|---|---|---|---|
| | wt | | | | % |
| | (average) | | | Yield | DOPA |
| Polymer | kDa | Color | Solubility | grams | by wt |
| PMA1 | 9.5 | White | Water | 2.11 | 0 |
| PMA2 | 11-12 | White | Water | 2.23 | 9.91% |
| PMA3 | 12-13 | White | Water | 2.56 | 22.24% |
| PBMA | 20-25 | Off white | Alkaline/ | 0.767 | 33.4% |
| | | | 0.5M HCl | | |
| PEMA | 600 | White | Alkaline/ | 0.964 | 39.9% |
| | | | 0.2M HCl | | |

These results demonstrated that polymers with desired high L-DOPA content can be created.

To create thin films, the poly-L-DOPA solution is layered on a stainless steel tray and frozen such that the crystal structure of the solution is sheet-like. The frozen solution is dried, and after sublimation and defrosting, the thin film is expected to retain its sheet structure. On one side of the film, agent solutions can be placed for delivery. The thin film can adhere to hair, nails, and/or skin due to its high L-DOPA content.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising:
an adhesive molecule comprising a plurality of 3,4-dihydroxyphenyl-L-alanine (DOPA) residues, and
metal ions or a dye attached to said adhesive molecule via an interaction with one or more of said DOPA residues,
wherein said adhesive molecule comprises the ability to interact with or bind to the hair, skin, or nails of a mammal.

2. The composition of claim 1, wherein said adhesive molecule is a mussel adhesive polypeptide.

3. The composition of claim 1, wherein said adhesive molecule is a polymer comprising a plurality of lysine residues and said plurality of DOPA residues.

4. The composition of claim 1, wherein said adhesive molecule is a polymer comprising a plurality of lysine residues, a plurality of glycine residues,. and said plurality of DOPA residues.

5. The composition of claim 1, wherein said adhesive molecule is a polymethacrylate polymer comprising said plurality of DOPA residues.

6. The composition of claim 1, wherein said composition comprises said metal ions, and wherein said metal ions are copper ions, bismuth ions, chromium ions, iron ions, silver ions, aluminum ions, manganese ions, zinc ions, or combinations thereof.

7. (canceled)

8. The composition of claim 1, wherein said composition comprises said dye, and wherein said dye is carmine, henna, guanine, pyrophyllite, or mica.

9. (canceled)

10. The composition of claim 1, said composition further comprising a polypeptide, and wherein said polypeptide is a keratin polypeptide or fluorescence emitting polypeptide.

11. (canceled)

12. The composition of claim 10, wherein said polypeptide is conjugated to said adhesive molecule.

13. The composition of claim 1, wherein said composition is a shampoo, hair conditioner, gel, polish, or paste.

14. A method for altering the appearance of hair, skin, or nails, wherein said method comprises:
(a) applying an adhesive molecule comprising a plurality of DOPA residues to hair, skin, or nails, wherein one or more of said DOPA resides interact with said hair, skin, or nails, and
(b) applying metal ions or a dye to said hair, skin, or nails, wherein said metal ions or said dye interact with one or more of said DOPA resides of said adhesive molecule,
wherein the appearance of said hair, skin, or nails is altered.

15. The method of claim 14, wherein said adhesive molecule and said metal ions or said dye are applied sequentially.

16. The method of claim 14, wherein said adhesive molecule and said metal ions or said dye are applied together.

17. The method of claim 14, wherein said adhesive molecule is selected from the group consisting of a polymethacrylate polymer, said polymer comprising said plurality of DOPA residues; a mussel adhesive polypeptide; a polymer comprising a plurality of lysine residues and said plurality of DOPA residues; and a polymer comprising a plurality of lysine residues, a plurality of glycine residues, and said plurality of DOPA residues.

18-20. (canceled)

21. The method of claim 14, wherein said adhesive molecule is a polymethacrylate polymer comprising said plurality of DOPA residues.

22. The method of claim 14, wherein said method comprises applying said metal ions to said hair, skin, or nails, and wherein said metal ions are copper ions, bismuth ions, chromium ions, iron ions, silver ions, aluminum ions, manganese ions, zinc ions, or combinations thereof.

23. (canceled)

24. The method of claim 14, wherein said method comprises applying said dye to said hair, skin, or nails, and wherein said dye is carmine, henna, guanine, pyrophyllite, or mica.

25. (canceled)

26. The method of claim 14, wherein said method further comprises applying a polypeptide to said hair, skin, or nails, and wherein said polypeptide is a keratin polypeptide or fluorescence emitting polypeptide.

27. (canceled)

28. The method of claim 26, wherein said polypeptide is conjugated to said adhesive molecule.

29. A method for altering the appearance of hair, skin, or nails of a mammal, wherein said method comprises applying said composition of claim 1 to hair, skin, or nails, wherein one or more of said DOPA resides interact with said hair, skin, or nails, and wherein the appearance of said hair, skin, or nails is altered.

30-39. (canceled)


SEQUENCE LISTING (37 sequences)

SEQ ID NO: 1 - DNA, 231 bp, Mytilus galloprovincialis
agttctgaagaatacaaaggtggttattacccaggcaatacttaccactatcattcaggtggtagttatcacggatccggctatcatggaggatataagggaaagtattacggaaaggcaaagaaatactattataaatataaaaacagcggaaaatacaagtatctgaagaaagctagaaaataccatagaaagggttacaagaagtattatggaggtggtagcagttag

SEQ ID NO: 2 - protein, 76 aa, Mytilus galloprovincialis
SSEEYKGGYYPGNTYHYHSGGSYHGSGYHGGYKGKYYGKAKKYYYKYKNSGKYKYLKKARKYHRKGYKKYYGGGSS

SEQ ID NO: 3 - protein, 98 aa, Mytilus edulis
MKLSCIVLVLFLVTLAAYSDVGSSSSEEYKGGYYPGNAYHYHSGGSYHGSGYHGGYKGKYYGKAKKYYYKYKNSGKYKYLKKARKYHRKGYKYYGGSS

SEQ ID NO: 4 - protein, 96 aa, Mytilus californianus
MKLSCVVLVLFLVTLAACIDVGSGYDGYSDGYYPGSAYNYPSGSHGYHGHGYKGKYYGKGKKYYYKYKRTGKYKYLKKARKYHRKGYKKYYGGGSS

SEQ ID NO: 5 - protein, 72 aa, Mytilus edulis
MNNTSVSVLVALVLIGSFAVQSDAADYYGPNYGPPRRYGGGNYNRYNRYGRRYGGYKGWNNGWNRGRRGKYW

SEQ ID NO: 6 - protein, 78 aa, Mytilus californianus
MNKFSVTVLLALVLIGFFAVQSDAGYGYDLGYDYYKGYNNPWPYGGKYNGYKGYPRGYGWNKGWNKGWNKGRWGRKYY

SEQ ID NO: 7 - protein, 70 aa, Mytilus galloprovincialis
MNNISVAVLVALVLIGSFAVQSDAADYYGPKYGPPRRYGGGNYNRYGRRYGGYKGWNNGWKRGRWGRKYY

SEQ ID NO: 8 - protein, 565 aa, Mytilus edulis
MEGIKLNLCLLCIFTFDVLGFSNGNIYNAHVSSYAGASAGAYKKLPNAYPYGTKPEPVYKPVKTSYSAPYKPPTYQQLKKKVDYRPTKSYPPTYGSKTNYLPLAKKLSSYKPIKTTYNAKTNYPPVYKPKMTYPPTYKPKPSYPPTYKSKPTYKPKITCPPTYKAKPSYPPTYKPKKTYPPTYKPKVTYPPTYKPKPSYPPIYKSKPTYKPKITYPPTYKAKPSYPPTYKAKPSYPPTYKAKPTYKAKPTYPSTYKAKPTYPPTYKAKPSYPPTYKAKPSYPPTYKAKPTYIAKPSYPPTYKAKPSYPPTYKAKPSYPPTYKAKSSYPPTYKAKPTYKAKPTYPSTYKAKPSYPPTYKAKPTYKAKPTYPSTYKAKPTYPSTYKAKPSYPPTYKPKISYPPTYKAKPSYPSTYKAKSSYPPTYKAKPSYPPTYKAKPTYKAKPTYPSTYKAKPTYKAKPTYPPTYKAKPSYPPTYKPKPSYPPTYKSKSSYPSSYKPKKTYPPTYKPKLTYPPTYKPKPSYPPSYKPKITYPSTYKLKPSYPPTYKSKTSYPPTYNKKISYPSQY

SEQ ID NO: 9 - protein, 732 aa, Mytilus californianus
MEGIKLNLCLLCIFSCDVFALSNGFIHNAYGSAYAGASAGAYKPLPGSYGSKHVPVYKPMNKIPTSYISKKSYPAPYKPKGYHPTNSYQPTYGSKTNYPPIYKPVAKKLSSYKAIKTTYLVYKAKTSYPPVYKHKITNPPTYKPKITYPPTYKPKPSYPPTYKPKPSYPPTYKAKKTYPSTYKPKPSYPPTYKPKITYPPTYKPKPSYPPSYKAKKSYPSTYKPKPSYPPTYKAKKTYPSTYKPKPSYPPTYKPKITYPPTYKPKPSYPPSYKAKKSYPSTYKPKPSYPPTYKPKITYPPTYKPKPSYPPTYKAKKTYPPTYKPKITYPPTYKPKPSYPTSYKSKKTYPPTYKPKITYPPTYKPKPSYPPSYKPKITYPPTYKPKKSYPPAYKSKASYPPSYQPKKTYLPSYKPKKTYPPTYKRKISYPPTYKTKPSYPSSYKRKTSYPSTYKRKTSYPPTYKPKISYPSTYKTKPSYPPTYKAKKTYPPTYKPKITYPPTYKPKPSYPTSYKSKKTYPPTYKPKITYPPTYKPKPSYPPSYKPKITYPPTYKPKKSYPPAYKSKASYPPSYQPKKTYLPSYKPKKTYPPTYKRKISYPPTYKTKPSYPASYKRKTSYPSTYKRKTSYPPTYKPKISYPSTYKTKPSYPPTYKPKPSYASSYKPKIRYPPTYKPKPSYASSYKPKITYPPTYKPKISYPPTYKPKITYPPSYKPKISYLPAYKPKISYPSQY

SEQ ID NO: 10 - DNA, 743 bp, Artificial Sequence
  synthetic nucleotide encoding a BFP polypeptide
ggatccaaggagatataacaatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctctcatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagagctc

SEQ ID NO: 11 - protein, 238 aa, Artificial Sequence
  synthetic BFP polypeptide
MSKGEELFTGVVPILVELDGDVNGHKFSVSGEGEGDATYGKLTLKFICTTGKLPVPWPTLVTTFSHGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 12 - protein, 234 aa, Artificial Sequence
  synthetic BFP polypeptide
MSEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 13 - protein, 10 aa, Mytilus edulis
  MOD_RES (6)...(7): can be Hyp
  MOD_RES 5, 9: can be DOPA
AKPSYPPTYK

SEQ ID NO: 14 - protein, 10 aa, Mytilus edulis
  MOD_RES (6)...(7): can be Hyp
  MOD_RES 5, 9: can be DOPA
PKISYPPTYK

SEQ ID NO: 15 - protein, 42 aa, Mytilus galloprovincialis
  MOD_RES 5, 29, 35, 37: can be DOPA
TDKAYKPNPCVVSKPCKNRGKCIWNGKAYRCKCAYGYGGRHC

SEQ ID NO: 16 - protein, 48 aa, Mytilus edulis
  MOD_RES 3, 4, 8, 14, 19, 22, 25, 29, 32, and/or 47: can be DOPA
ADYYGPNYGPPRRYGGGNYNRYNRYGRRYGGYKGWNNGWNRGRRGKYW

SEQ ID NO: 17 - protein, 10 aa, Mytilus californianus
HVHTHRVLHK

SEQ ID NO: 18 - protein, 11 aa, Mytilus californianus
DDHVNDIAQTA

SEQ ID NO: 19 - protein, 73 aa, Artificial Sequence
  mussel adhesive polypeptide consensus sequence
  MOD_RES 5, 9, 10, 15, 17, 22, 27, 31, 35, 36, 42, 43, 44, 46, 52,54, 61, 66, 68, and/or 69: can be DOPA
SSEEYKGGYYPGNAYHYSGGSYHGSGYHGGYKGKYYGKAKKYYYKYKNSGKYKYLKKARKYHRKGYKYYGGSS

SEQ ID NO: 20 - protein, 99 aa, Artificial Sequence
  mussel adhesive polypeptide consensus sequence
  MOD_RES 5, 8, 18, 21, 29, 34, 36, 41, 49, 54, 55, 60, 67, 68, 69,74, 75, 76, 77, 82, 90, and/or 99: can be DOPA
GGGNYRGYCSNKGCRSGYIFYDNRGFCKYGSSSYKYDCGNYACLPRNPYGRVKYYCTKKYSCPDDFYYYNNKGYYYYNDKDYGCFNCGSYNGCCLRSGY

SEQ ID NO: 21 - protein, 50 aa, Artificial Sequence
  synthetic adhesive molecule
  MOD_RES 2, 7, 11, 15, 16, 22, 23, 24, 26, 32, 34, 41, 46, 48, 49: Xaa = L-DOPA
KXKGKGXKGGXKGKXXGKGKKXXXKXKGKGKXKXGKKKGKXKGKGXKXXG

SEQ ID NO: 22 - protein, 50 aa, Artificial Sequence
  synthetic adhesive molecule
  MOD_RES 2, 7, 11, 15, 16, 22, 23, 24, 26, 32, 34, 41, 46, 48, 49: Xaa = L-DOPA
KXKKKKXKKKXKKKXXKKKKKXXXKXKKKKKXKXKKKKKKXKKKKXKXXK

SEQ ID NO: 23 - protein, 50 aa, Artificial Sequence
  synthetic adhesive molecule
  MOD_RES 2, 7, 11, 15, 16, 22, 23, 24, 26, 32, 34, 41, 46, 48, 49: Xaa = L-DOPA
SXHGSGXHGGXKGKXXGKAKKXXXKXKNSGKXKXLKKARKXHRKGXKXXG

SEQ ID NO: 24 - protein, 50 aa, Artificial Sequence
  synthetic adhesive molecule
  MOD_RES 2, 7, 11, 15, 16, 22, 23, 24, 26, 32, 34, 41, 46, 48, 49: Xaa = L-DOPA
SXSSSSXSSSXKSKXXSKSKKXXXKXKSSSKXKXSKKSSKXSSKSXKXXS

SEQ ID NO: 25 - protein, 50 aa, Artificial Sequence
  synthetic adhesive molecule
  MOD_RES 2, 7, 11, 15, 16, 22, 23, 24, 26, 32, 34, 41, 46, 48, 49: Xaa = L-DOPA
GXSGKNXHGSXKGKXXHKHKKXXXKXKLLHKXKXGKKGNKXGGKGXKXXH

SEQ ID NO: 26 - protein, 50 aa, Artificial Sequence
  synthetic adhesive molecule
  MOD_RES 2, 4, 6, 7, 9, 10, 11, 13, 15, 16, 17, 19, 22, 23, 24, 26,28, 30, 32, 34, 35, 39, 41, 43, 45, 46, 48, 49, 50: Xaa = L-DOPA
KXKXKXXKXXXKXKXXXKXKKXXXKXKXKXKXKXXKKKXKXKXKXXKXXX

SEQ ID NO: 27 - protein, 50 aa, Artificial Sequence
  synthetic adhesive molecule
  MOD_RES 2, 11, 16, 23, 26, 34, 46, 49: Xaa = L-DOPA
KXKGKGGKGGXKGKGXGKGKKGXGKXKGKGKGKXLKKKGKGKGKGXKGXG

SEQ ID NO: 28 - protein, 50 aa, Artificial Sequence
  synthetic adhesive molecule
  MOD_RES 2, 7, 11, 15, 16, 22, 23, 24, 32, 34, 41, 46, 48, 49: Xaa = L-DOPA
KXKKKKXKKKXKKKXXKKKKKXXXKYKKKKKXKXKKKKKKXKKKKXKXXK

SEQ ID NO: 29 - protein, 50 aa, Artificial Sequence
  synthetic adhesive molecule
  MOD_RES 5, 6, 10, 12, 20, 21, 25, 26, 31, 34, 35, 36, 37, 38, 42,43, 45, 47: Xaa = L-DOPA
KKGGXXKGKXKXGKKKKGGXXGGKXXGKKKXGKXXXXXKGKXXGXKXGKK

SEQ ID NO: 30 - protein, 5 aa, Artificial Sequence
  synthetic adhesive molecule
  MOD_RES 3, 4: Xaa = L-DOPA
GKXXG

SEQ ID NO: 31 - protein, 10 aa, Artificial Sequence
  synthetic adhesive molecule
  MOD_RES 3, 8: Xaa = L-DOPA
GKXGGGKXGG

SEQ ID NO: 32 - protein, 15 aa, Artificial Sequence
  synthetic adhesive molecule
  MOD_RES 2, 3, 9, 10, 11, 13: Xaa = L-DOPA
KXXGKGKKXXXKXKG

SEQ ID NO: 33 - protein, 13 aa, Artificial Sequence
  synthetic adhesive molecule
  MOD_RES 2, 3, 9, 10, 11: Xaa = L-DOPA
KXXGKGKKXXXKG

SEQ ID NO: 34 - protein, 13 aa, Artificial Sequence
  synthetic adhesive molecule
  MOD_RES 3, 9, 10, 11: Xaa = L-DOPA
KGXGKGKKXXXKG

SEQ ID NO: 35 - protein, 13 aa, Artificial Sequence
  synthetic adhesive molecule
  MOD_RES 9, 10, 11: Xaa = L-DOPA
KGKGKGKKXXXKG

SEQ ID NO: 36 - protein, 10 aa, Artificial Sequence
  synthetic adhesive molecule
  MOD_RES 2, 5, 8: Xaa = L-DOPA
KXGKXGKXKG

SEQ ID NO: 37 - protein, 50 aa, Artificial Sequence
  adhesive polypeptide consensus sequence
  MOD_RES 1, 3-6, 8-10, 12-14, 17-21, 25, 27-31, 33, 35-40, 42-45,47, 50: Xaa = Lys, Gly, Ser, His, Asn
  MOD_RES 2, 7, 11, 15-16, 22-24, 26, 32, 34, 41, 46, 48-49: Xaa = DOPA
XXXXXXXXXXXXXXXXXXXXXXXXXXXXXXXXXXXXXXXXXXXXXXXXXX